(19) United States
(12) Patent Application Publication
(10) Pub. No.: US 2023/0263757 A1
(43) Pub. Date: Aug. 24, 2023

(54) DISINFECTING WIPE KIT FOR CENTRAL VENOUS ACCESS DEVICES

(71) Applicant: SterileCare Inc., Markham (CA)

(72) Inventor: Karen Mueller, Newmarket (CA)

(21) Appl. No.: 18/112,625

(22) Filed: Feb. 22, 2023

Related U.S. Application Data
(60) Provisional application No. 63/312,516, filed on Feb. 22, 2022.
(60) Provisional application No. 63/312,628, filed on Feb. 22, 2022.
(60) Provisional application No. 63/396,052, filed on Aug. 8, 2022.
(60) Provisional application No. 63/312,501, filed on Feb. 22, 2022.
(60) Provisional application No. 63/312,509, filed on Feb. 22, 2022.

(51) Int. Cl. A61K 31/198; A61K 31/155; A61K 31/045

(52) U.S. Cl. CPC A61K 31/198; A61K 31/155; A61K 31/045; A61M 2025/0019

(57) ABSTRACT

Provided herein are compositions and solutions containing tetrasodium EDTA, ethanol, and chlorhexidine, and optionally taurolidine, heparin, or a thrombolytic agent. The compositions may be disposed on a cleaning wipe to provide broad-spectrum antimicrobial activity on planktonic and biofilm cells of clinically relevant pathogens. Also provided herein is a peripheral line placement kit containing the compositions and solutions.

### CROSS-REFERENCE TO RELATED APPLICATIONS

This application claims priority to U.S. Provisional Application No. 63/312,516 entitled “SYNERGISTIC ACTIVITY OF TETRASODIUM EDTA, ETHANOL, AND CHLORHEXIDINE HYDROCHLORIDE AGAINST PLANKTONIC AND BIOFILM CELLS OF CLINICALLY RELEVANT PATHOGENS”, filed Feb. 22, 2022, and to U.S. Provisional Application No. 63/312,628 entitled “SYNERGISTIC ACTIVITY OF TETRASODIUM EDTA AND HEPARIN AGAINST PLANKTONIC AND BIOFILM CELLS OF CLINICALLY RELEVANT PATHOGENS”, filed Feb. 22, 2022, and to U.S. Provisional Application No. 63/396,052 entitled “MULTIPURPOSE SOLUTION FOR IMPROVED CATHETER LOCKS OR ENHANCED SAFETY OF IMPLANTABLE MEDICAL DEVICES”, filed Aug. 8, 2022, and to U.S. Provisional Application No. 63/312,501 entitled “DISINFECTING WIPE KIT WITH TETRASODIUM EDTA, ETHANOL, AND CHLORHEXIDINE”, filed Feb. 22, 2022, and to U.S. Provisional Application No. 63/312,509 entitled “PERIPHERAL LINE DISINFECTING KIT WITH TETRASODIUM EDTA, ETHANOL, AND CHLORHEXIDINE”, filed Feb. 22, 2022, the entire contents of each of which are incorporated by reference herein.

### FIELD OF THE INVENTION

The present invention generally relates to compositions and solutions from the combination of tetrasodium EDTA, ethanol, and chlorhexidine, which has broad-spectrum antimicrobial activity on planktonic and biofilm cells of clinically relevant pathogens. The invention also relates to cleaning solutions, cleaning wipes, and kits containing such compositions and solutions.

### BACKGROUND OF THE INVENTION

Biofilms associated with implantable medical devices and wounds are clinically relevant, often requiring repeated antibiotics without success.

The number of patients predisposed to hospital-acquired infections has been on the rise owing to an increase in patients with impaired immunity and chronic diseases and the administration of immunosuppressants or anticancer agents. Patients in the intensive care unit (ICU) are more susceptible to hospital-acquired infections than those in general wards and are susceptible to infection with pathogenic micro-organisms through various implantable medical devices. In particular, central venous access devices (CVADs) are among the most common sources of healthcare-associated bloodstream infections worldwide, with a mortality rate of 12-25%. The use of long-term CVADs is inevitable for patients admitted in nephrology, oncology, and ICUs owing to the ease of administration of blood products, fluids, parenteral nutrients, and medical therapies to the blood¬stream. Unfortunately, CVADs are prone to complications such as occlusion, clot formation, and microbial colonization, all of which lead to prolonged hospitalization, expensive treatments, and significant mortality and morbidity.

Biofilms formed within CVADs are resistant to systemic antibiotic therapy alone, with 10- to 1000-fold greater resistance to conventional antibiotics than planktonic cells. Appropriate control measures and management of catheter-related infections have become a significant challenge for physicians.

To salvage long-term CVADs, the use of antimicrobial lock solutions (ALSs) has been proposed in addition to parenteral administration of antibiotics for the prevention and treatment of central line bloodstream infections (CLABSIs). Catheter lumens may be locked with highly concentrated antibiotic solutions and allowed to dwell for a specified time to fight biofilm formation. However, the prophylactic use of antibiotic locks increases concerns about the emergence of multidrug resistance among pathogens. There is clearly a need for improved lock solutions, lock flushes, catheter systems, wipes, cleaning agents, and other products for reducing infection in health care settings such as catheter use and in a variety of other settings.

There is a need in the art for improved solutions and compositions thereof for eliminating or reducing planktonic and biofilm cells of clinical relevant pathogens.

### SUMMARY OF THE INVENTION

Provided herein is a cleaning wipe kit. The kit may comprise at least one first wipe and at least one second wipe. The first wipe may comprise a first composition. The first composition may comprise 1-15% (w/v) tetrasodium EDTA and ethanol. The first composition may have a pH of at least 9.5. The first composition may comprise 10-60% (w/v) ethanol. The first composition may comprise 3% (w/v) tetrasodium EDTA and 20-25% (w/v) ethanol. The first composition may comprise 20% (w/v) ethanol.

The second wipe may comprise a second composition. The second composition may comprise 1.0-15% (w/v) tetrasodium EDTA, ethanol, and chlorhexidine. The second composition may have a pH of at least 9.5. The second composition may comprise 10-60% (w/v) ethanol. The second composition may comprise 0.5-6% (w/v) chlorhexidine. The second composition may comprise 3% (w/v) tetrasodium EDTA, 20-25% (w/v) ethanol, and 1-2% (w/v) chlorhexidine. The second composition may comprise 20% (w/v) ethanol.

The at least one first wipe may exude the first composition and the at least one second wipe may exude the second composition. The cleaning wipe kit may be associated with a catheter. The at least one first wipe may be associated with insertion of a catheter. The at least one second wipe may be associated with service of a catheter.

Further provided herein is a peripheral line placement kit. The kit may comprise a peripheral line and a solution chamber in communication with the peripheral line. The solution chamber may be configured to dispense a first disinfecting solution. The first disinfecting solution may comprise 1-15% (w/v) tetrasodium EDTA, ethanol, chlorhexidine, and at least 1% (w/v) taurolidine. The first disinfecting solution may have a pH of 6.5-7.5 and may be biocompatible in a bloodstream.

The peripheral line placement kit may comprise at least one additional product. The at least one additional product may be a flush solution, a lock solution, a swab, a wet wipe, or a cleaning product. The at least one additional product may comprise a second disinfecting solution. The second disinfecting solution may comprise at least three of tetrasodium EDTA, ethanol, taurolidine, and chlorhexidine. A composition of the second disinfecting solution may be different from a composition of the first disinfecting solution.

### BRIEF DESCRIPTION OF THE DRAWINGS

FIG. 1 illustrates an exemplary catheter cleaning kit.

FIG. 2 illustrates an exemplary wipe dispenser.

FIG. 3 illustrates an exemplary two-composition wipe dispenser.

FIG. 4 illustrates an exemplary peripheral line kit.

FIG. 5 illustrates an exemplary pre-filled syringe.

FIGS. 6A-6B illustrate an exemplary determination of the minimum biofilm eradication concentration (MBEC) for tetrasodium EDTA against gram-positive, gram-negative, and fungal biofilms. CFU/mL were enumerated from each peg (n=8) after biofilm growth for 48 h and following antimicrobial exposure for 24 h, where points on the graph represent the mean±standard deviation from three independent experiments, and statistical significance is indicated as follows: *: P<0.05; **: P<0.005; ***: P<0.0005; ****: P<0.0001.

FIGS. 7A-7B illustrate an exemplary determination of the minimum biofilm eradication concentration (MBEC) for ethanol against gram-positive, gram-negative, and fungal biofilms CFU/ml were enumerated from each peg (n=8) after biofilm growth for 48 h and following antimicrobial exposure for 24 h, where points on the graph represent the mean±standard deviation from three independent experiments, and statistical significance is indicated as follows: *: P<0.05; **: P<0.005; ***: P<0.0005; ****: P<0.0001.

FIGS. 8A-8B illustrate an exemplary determination of the minimum biofilm eradication concentration (MBEC) for chlorhexidine HCl against gram-positive, gram-negative, and fungal biofilms. CFU/mL were enumerated from each peg (n=8) after biofilm growth for 48 h and following antimicrobial exposure for 24 h, where points on the graph represent the mean±standard deviation from three independent experiments, and statistical significance is indicated as follows: *: P<0.05; **: P<0.005; ***: P<0.0005; ****: P<0.0001.

FIGS. 9A-9C illustrate an exemplary efficacy of tetrasodium EDTA (TE), ethanol (ET), and chlorhexidine HCl (CH) against gram-positive and gram-negative bacterial biofilms in a 2-h exposure period, where each column represents different concentrations of test antimicrobials against each organism tested: (i) three dark grey columns represent treatment with the MBEC of TE (%), ET (%) and CH (μg/mL); (ii) three light grey column s represents treatment with the FBEC of TE+ET, TE+CH, and TE+ET+CH; (iii) white and hatched columns represent treatment with triple combinations of TE+ET+CH, with hatched bar combinations showing the best killing effects, and statistical significance was determined by comparison with non-treated biofilms (black bar) and is indicated as follows: *: P<0.05; **: P<0.005; P<0.0005; P<0.0001. MBEC=minimum biofilm eradication concentration; FBEC=fractional biofilm eradication concentration.

FIG. 10 illustrates an exemplary efficacy of tetrasodium EDTA (TE), ethanol (ET), and chlorhexidine HCl (CH) against fungal biofilms in a 2-h exposure period, where each column represents different concentrations of test antimicrobials against each organism tested: (i) three dark grey columns represent treatment with the MBEC of TE (%), ET (%) and CH (μg/mL); (ii) three light grey column represents treatment with the FBEC of T E+ET, TE+CH, and TE+ET+CH; (iii) white and hatched columns represent treatment with triple combinations of TE+ET+CH, with hatched bar combinations showing the best killing effects, and statistical significance was determined by comparison with non-treated biofilms (black bar) and is indicated as follows: *: p<0.05; **: p<0.005. MBEC, minimum biofilm eradication concentration; FBEC, fractional biofilm eradication concentration.

### DETAILED DESCRIPTION

Aspects disclosed herein are disclosed in the following description and related figures directed to specific embodiments of the invention. Those of ordinary skill in the art will recognize that alternate embodiments may be devised without departing from the claims' spirit or scope. Additionally, well-known elements of exemplary embodiments of the invention will not be described in detail or will be omitted so as not to obscure the relevant details of the invention. As used herein, the word exemplary means serving as an example, instance, or illustration. The embodiments described herein are not limiting but rather are exemplary only. It should be understood that the described embodiments are not necessarily to be construed as preferred or advantageous over other embodiments. Moreover, the terms embodiments of the invention, embodiments, or invention do not require that all embodiments include the discussed feature, advantage, or mode of operation.

The formulations of the present disclosure are safe for human administration and are biocompatible and non-corrosive. They may also have anticoagulant properties and are thus useful for preventing and/or treating a variety of catheter-related infections. The antiseptic solutions of disclosed herein have numerous applications, including lock and lock flush solutions for various types of catheters and are used as antiseptic agents or solutions for sanitizing a range of medical, dental, and veterinary devices instruments and other objects, surfaces, and the like. They furthermore have sanitizing applications in industrial and food preparation and handling settings.

The formulations may be in the form of solutions, wherein the solvent or carrier comprises water or saline. Preferably, the composition comprises water, such as purified, distilled, double-distilled, or deionized water, or water for injection.

In one embodiment, antiseptic compositions are disclosed that have at least four, and preferably at least five, of the following properties: anticoagulant properties; inhibitory and/or bactericidal activity against a broad spectrum of bacteria in a planktonic form; inhibitory and/or fungicidal activity against a spectrum of fungal pathogens; inhibitory and/or bactericidal activity against a broad spectrum of bacteria in a sessile form; inhibitory activity against protozoan infections; inhibitory activity against Acanthamoeba infections; safe and biocompatible, at least in modest volumes, in contact with a patient; safe and biocompatible, at least in modest volumes, in a patient's bloodstream; and safe and compatible with industrial objects and surfaces.

Importantly, in most embodiments, sanitizing compositions and methods of the present disclosure do not comprise traditional antibiotic agents (e.g., beta-lactams, aminoglycosides, chloramphenicol, glycopeptides, quinolones, oxazolidinones, sulfonamides, tetracyclines, macrolides, ansamycins, streptogramins, lipopeptides, etc.) and thus do not contribute to the development of antibiotic-resistant organisms.

Methods for inhibiting the growth and proliferation of microbial populations and/or fungal pathogens, including inhibiting the formation and proliferation of biofilms, are provided that comprise contacting an infected or suspected infected object, or surface, with a sanitizing composition disclosed herein. Methods for inhibiting the growth and proliferation of protozoan populations are provided, comprising contacting an infected or suspected infected object, or surface, with a sanitizing composition disclosed herein. Methods for inhibiting the growth and proliferation of amoebic populations and preventing amoebic infection, particularly Acanthamoeba infections, are provided, comprising contacting an object, or a surface, with a sanitizing composition disclosed herein.

Methods for substantially eradicating microbial populations, including both planktonic microbial populations and microbial populations in the form of biofilms, are also provided and comprise contacting an infected or suspected infected object, or surface, with a sanitizing composition of the present disclosure. Depending on the antiseptic composition used in the various methods, various compositions and contact time periods may be required to inhibit the formation and proliferation of various populations and/or to substantially eradicate various populations. Suitable contact time periods for various compositions are provided in the examples and may be determined by routine experimentation.

Soluble salts of EDTA are used in compositions disclosed herein. Sodium salts of EDTA are commonly available and generally used, including di-sodium, tri-sodium, and tetra-sodium salts. However, other EDTA salts, including ammonium, di-ammonium, potassium, di-potassium, cupric di-sodium, magnesium di-sodium, ferric sodium, and combinations thereof, may be used, provided they have the antibacterial and/or fungicidal and/or anti-protozoan and/or anti-amoebic properties desired, and provided that they are sufficiently soluble in the solvent desired. Various combinations of EDTA salts may be used and may be preferred for particular applications. Importantly, in most embodiments, sanitizing compositions and methods disclosed herein do not employ traditional antibiotic agents and thus do not contribute to the development of antibiotic-resistant organisms.

In one embodiment, antiseptic compositions consisting of, consisting essentially of, or comprising one or more sodium salt(s) of EDTA at a pH higher than physiological pH are provided as antiseptic compositions disclosed herein. The concentration of EDTA in the composition may be from about 0.5% (w/v) to about 15% (w/v), such as from about 0.5% (w/v) to about 2.5% (w/v), about 1.0% (w/v) to about 5.0% (w/v), about 1.0% (w/v) to about 7.5% (w/v), about 1.0% (w/v) to about 10% (w/v), about 1.0% (w/v) to about 12.5% (w/v), about 1.0% (w/v) to about 15% (w/v), about 2.5% (w/v) to about 15% (w/v), about 5.0% (w/v) to about 15% (w/v), about 7.5% (w/v) to about 15% (w/v), about 10% (w/v) to about 15% (w/v), or about 12.5% (w/v) to about 15% (w/v). Therefore, the concentration of EDTA in the composition may be about 1.0% (w/v), about 2.0% (w/v), about 3.0% (w/v), about 4.0% (w/v), about 5.0% (w/v), about 6.0% (w/v), about 7.0% (w/v), about 8.0% (w/v), about 9.0% (w/v), about 10% (w/v), about 11% (w/v), about 12% (w/v), about 13% (w/v), about 14% (w/v), or about 15% (w/v). Preferably, the EDTA has a concentration of at least about 1% (w/v).

In other embodiments, the EDTA may have a concentration in the composition from about 0.015% (w/v) to about 2% (w/v). For example, the EDTA may have a concentration in the composition from about 0.015% (w/v) to about 0.05% (w/v), about 0.015% (w/v) to about 0.1% (w/v), about 0.015% (w/v) to about 0.5% (w/v), about 0.015% (w/v) to about 1% (w/v), about 0.015% (w/v) to about 1.5% (w/v), about 0.015% (w/v) to about 2% (w/v), about 0.05% (w/v) to about 2% (w/v), about 0.1% (w/v) to about 2% (w/v), about 0.5% (w/v) to about 2% (w/v), about 1% (w/v) to about 2% (w/v), or about 1.5% (w/v) to about 2% (w/v). Therefore, the concentration of EDTA in the composition may be about 0.015% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.5% (w/v), or about 2% (w/v).

The pH may be higher than 6.5, such as 6.5-9.5, 6.5-8.5, or 6.5-7.5. The antiseptic compositions have application as lock solutions and lock flush solutions for various types of in-dwelling access catheters, including vascular catheters used for delivery of fluids, blood products, drugs, nutrition, withdrawal of fluids or blood, dialysis, monitoring of patient conditions, and the like. In some examples, the antiseptic solutions have applications in peripheral catheters including peripheral IVs, peripherally inserted central catheters (PICC lines), and other peripheral lines. Antiseptic solutions disclosed herein may also be used as lock and lock flush solutions for urinary catheters, nasal tubes, throat tubes, and the like. The general solution parameters described below are suitable for these purposes. In one embodiment, an antiseptic solution consisting of, consisting essentially of, or comprising one or more sodium EDTA salt(s) at a pH higher than physiological pH is provided to maintain the patency of in-dwelling intravascular access devices. In one example, the pH is higher than 6.5. Methods for sanitizing catheters and other medical tubes, such as nasal tubes, throat tubes, and the like, are also provided and involve contacting the catheter or other medical tube with a sanitizing composition disclosed herein.

The composition may further comprise ethanol. The ethanol may be present at a concentration from about 0.1% (w/v) to about 70% (w/v). For example, the ethanol may have a concentration in the composition from about 0.1% (w/v) to about 1% (w/v), about 0.1% (w/v) to about 5% (w/v), about 0.1% (w/v) to about 10% (w/v), about 0.1% (w/v) to about 30% (w/v), about 0.1% (w/v) to about 50% (w/v), about 0.1% (w/v) to about 70% (w/v), about 1% (w/v) to about 70% (w/v), about 5% (w/v) to about 70% (w/v), about 10% (w/v) to about 70% (w/v), about 30% (w/v) to about 70% (w/v), about 50% (w/v) to about 70% (w/v), about 5% (w/v) to about 70% (w/v), about 10% (w/v) to about 50% (w/v), about 10% (w/v) to about 40% (w/v), about 10% (w/v) to about 30% (w/v), about 5% (w/v) to about 20% (w/v), or about 3.125% (w/v) to about 12.5% (w/v). Further, the composition may comprise ethanol in a concentration of about 0.1% (w/v), about 0.5% (w/v), about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), about 20% (w/v), about 30% (w/v), about 40% (w/v), about 50% (w/v), about 60% (w/v), or about 70% (w/v).

The composition may further comprise chlorhexidine or a pharmaceutically acceptable salt thereof. Other compositions and solutions derived from chlorhexidine[1,6-bis(4′-chlorophenyl biguanide) hexane] are divalent cationic biguanide agents that exist as acetate, gluconate, and hydrochloride salts. In preferred embodiments when the composition comprises chlorhexidine, the composition comprises chlorhexidine HCl. The chlorhexidine may have a concentration in the composition from about 0.5% (w/v) to about 6% (w/v), such as from about 0.5% (w/v) to about 1% (w/v), about 0.5% (w/v) to about 2% (w/v), about 0.5% (w/v) to about 3% (w/v), about 0.5% (w/v) to about 4% (w/v), about 0.5% (w/v) to about 5% (w/v), about 0.5% (w/v) to about 6% (w/v), about 1% (w/v) to about 6% (w/v), about 2% (w/v) to about 6% (w/v), about 3% (w/v) to about 6% (w/v), about 4% (w/v) to about 6% (w/v), about 5% (w/v) to about 6%, about 1% (w/v) to about 3% (w/v), or about 1% (w/v) to about 2% (w/v). In other embodiments, the composition may be free of chlorhexidine or may be substantially free of chlorhexidine (i.e., less than 0.01 μg/mL).

The composition may further include taurolidine. The taurolidine may be present in the composition at a concentration from about 0.5% (w/v) to about 8% (w/v). For example, the taurolidine may be present at a concentration from about 0.5% (w/v) to about 1% (w/v), about 0.5% (w/v) to about 2% (w/v), about 0.5% (w/v) to about 3% (w/v), about 0.5% (w/v) to about 4% (w/v), about 0.5% (w/v) to about 5% (w/v), about 0.5% (w/v) to about 6% (w/v), about 0.5% (w/v) to about 7% (w/v), about 0.5% (w/v) to about 8% (w/v), about 1% (w/v) to about 8% (w/v), about 1.5% (w/v) to about 8% (w/v), about 2% (w/v) to about 8% (w/v), about 3% (w/v) to about 8% (w/v), about 4% (w/v) to about 8% (w/v), about 5% (w/v) to about 8% (w/v), about 6% (w/v) to about 8% (w/v), about 7% (w/v) to about 8% (w/v), about 2% (w/v) to about 7% (w/v), about 1% (w/v) to about 6% (w/v), or about 1% (w/v) to about 4% (w/v). The taurolidine may be present in the composition at a concentration of about 0.5% (w/v), about 1% (w/v), about 1.5% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), or about 8% (w/v). In other embodiments, the composition may be free of taurolidine or substantially free of taurolidine (i.e., less than 0.01% w/v taurolidine).

The composition may include heparin, heparan sulfate, or a combination thereof in a concentration of at least about 0.5% (w/v). The heparin may be present in a concentration from about 1% (w/v) to about 8% (w/v), such as from about 0.5% (w/v) to about 1% (w/v), about 0.5% (w/v) to about 2% (w/v), about 0.5% (w/v) to about 3% (w/v), about 0.5% (w/v) to about 4% (w/v), about 1% (w/v) to about 5% (w/v), about 0.5% (w/v) to about 6% (w/v), about 0.5% (w/v) to about 7% (w/v), about 0.5% (w/v) to about 8% (w/v), about 2% (w/v) to about 8% (w/v), about 3% (w/v) to about 8% (w/v), about 4% (w/v) to about 8% (w/v), about 5% (w/v) to about 8% (w/v), about 6% (w/v) to about 8% (w/v), about 7% (w/v) to about 8% (w/v), about 1.5% (w/v) to about 8% (w/v), about 2% (w/v) to about 7% (w/v). In some exemplary embodiments, the heparin may be present in a concentration from about 0.5% (w/v) to about 1.8% (w/v), about 1% (w/v) to about 2.5% (w/v), or from about 0.5% (w/v) to about 4% (w/v).

The heparin, heparan sulfate, or combination thereof may further be present in a concentration of about 0.5% (w/v), about 1% (w/v), about 1.5% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), or about 8% (w/v), or about 1% (w/v) to about 8% (w/v). In another embodiment, the composition may include heparin in a concentration of at least about 0.5% (w/v), at least about 1% (w/v), at least about 2% (w/v), at least about 5% (w/v), or at least about 8% (w/v). Preferably, the heparin has a concentration of at least about 1%.

In still further embodiments, the composition may be free of heparin and/or heparan sulfate or substantially free of heparin and/or heparan sulfate (i.e., less than 0.01% w/v heparin and/or heparan sulfate).

In additional embodiments, the composition may include heparin in a weight ratio of EDTA to heparin from about 0.025:1 to about 40:1. For example, the weight ratio of EDTA to heparin may be from about 0.025:1 to about 0.1:1, about 0.025:1 to about 0.5:1, about 0.025:1 to about 1:1, about 0.025:1 to about 2:1, about 0.025:1 to about 5:1, about 0.025:1 to about 10:1, about 0.025:1 to about 25:1, about 0.025:1 to about 40:1, about 0.1:1 to about 40:1, about 0.5:1 to about 40:1, about 1:1 to about 40:1, about 2:1 to about 40:1, about 5:1 to about 40:1, about 10:1 to about 40:1, about 25:1 to about 40:1, about 0.2:1 to about 5:1, about 0.1:1 to about 1:1, about 1:1 to about 10:1, or about 0.5:1 to about 20:1. In some additional embodiments, the composition may include heparin in a weight ratio of EDTA to heparin of about 0.025:1, 0.05:1, 0.075:1, 0.1:1, 0.25:1, 0.5:1, 0.75:1, 1:1, 1.5:1, 2:1, 3:1, 4:1, 5:1, 10:1, 15:1, 20:1, 25:1, 30:1, 35:1, or about 40:1.

The composition may further comprise a thrombolytic agent. Thrombolytic agents such as alteplase, urokinase, and streptokinase may be considered to deal with existing clots that cause thrombosis or occlude catheters. Such agents and their mechanisms are unrelated to EDTA's antibacterial or anti-biofilm activity and may require different conditions than those that provide optimum performance of EDTA in a catheter lock solution. Despite multiple barriers, the Applicant has examined the possibility of an unexpected synergistic effect between EDTA and thrombolytic compounds, such that novel products and methods based on combining both classes of compounds can now be provided for improved results with implantable medical devices such as catheters. Such synergistic effects may include, but are not limited to, enhanced efficacy in preventing blood clots or undermining existing clots, enhanced efficacy in biofilm mitigation or prevention, enhanced stability or lifetime of a thrombolytic agent or solution comprising a thrombolytic agent, reduced requirement for system use of thrombolytic agents in association with implantable medical devices, etc.

The thrombolytic agent may comprise a protein or protein mixture, and more particularly may comprise an enzyme or a mixture of enzymes. In some aspects, however, the thrombolytic agent does not include heparin or aspirin. In preferred embodiments, the thrombolytic agent may comprise one or more enzymes such as alteplase, streptokinase, reteplase, tenecteplase, urokinase, prourokinase, anistreplase (APSAC), etc.

“Alteplase” is a complex fibrinolytic agent, an enzyme, that is manufactured from recombinant DNA. Sometimes it is referred to as a tissue plasminogen activator (tPA). Alteplase converts plasminogen to the proteolytic enzyme plasmin, which can lyse fibrin and fibrinogen. It is often provided commercially as a lyophilized powder in, for example, 50 mg and 100 mg vials. Each vial may be packaged with diluent (e.g., sterile water for injection) for reconstitution. It is compatible with 0.9% sodium chloride (NS) and dextrose 5% water (D5W).

“Streptokinase” is an enzyme, a purified fibrinolytic bacterial protein used to break down thrombosis in situations such as myocardial infarction, pulmonary embolism, and venous thromboembolism.

“Urokinase,” also known as urokinase-type plasminogen activator (uPA), is a serine protease present in humans and other animals. It can be described as a trypsin-like enzyme that is produced endogenously by renal parenchymal cells.

“Reteplase” may also be considered. Reteplase is a recombinant tissue plasminogen activator and modified nonglycosylated form of tPA used to dissolve intracoronary emboli, promote lysis of acute pulmonary emboli, and assist the handling of myocardial infarction. Reteplase catalyzes the cleavage of endogenous plasminogen to generate plasmin. Plasmin degrades the fibrin matrix of the thrombus. Reteplase is indicated for treating acute ST-elevation myocardial infarction (STEMI) to reduce the risk of death and heart failure.

“Prourokinase” is a relatively inactive precursor that requires the conversion to urokinase to become active.

“Tenecteplase” (TNK-tPA) is a commonly used fibrinolytic agent said to be as efficient as alteplase while exerting a lower risk of non-cerebral bleeding. Tenecteplase has higher fibrin specificity and a longer plasma half-life with final clearance, mostly through hepatic metabolism.

“Anistreplase is an anisoylated purified streptokinase activator complex (APSAC), a complex mixture of streptokinase and plasminogen that does not depend on circulating plasminogen to be effective.

Other known thrombolytic agents may be considered if they become approved for human or animal use. Such thrombolytic agents include, for example, Desmoteplase, a highly fibrin-specific thrombolytic experimental drug.

In preferred embodiments, the thrombolytic agent may comprise alteplase, urokinase, streptokinase, or a combination thereof.

The thrombolytic agent may be present in the composition at a concentration from about 0.01% (w/v) to about 1.5% (w/v). For example, the thrombolytic agent may be present in the composition at a concentration from about 0.01% to about 0.05% (w/v), about 0.01% (w/v) to about 0.1% (w/v), about 0.01% (w/v) to about 0.5% (w/v), about 0.01% (w/v) to about 1% (w/v), about 0.01% (w/v) to about 1.5% (w/v), about 0.05% (w/v) to about 1.5% (w/v), about 0.1% (w/v) to about 1.5% (w/v), about 0.5% (w/v) to about 1.5% (w/v), about 1% (w/v) to about 1.5% (w/v), or about 0.03% (w/v) to about 1.5% (w/v). In additional embodiments, the thrombolytic agent may be present in the composition at a concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), or about 1.5% (w/v). In other embodiments, the composition may be free of a thrombolytic agent, or may be substantially free of a thrombolytic agent (i.e., less than 0.001% (w/v))

The composition may further comprise a surfactant, such as a cationic surfactant, an anionic surfactant, a zwitterionic surfactant, or combinations thereof. The surfactant may be a present at a concentration from about 0.5% (w/v) to about 20% (w/v). For example, the surfactant may be present at a concentration from about 0.1% (w/v) to about 5% (w/v), about 0.1% (w/v) to about 10% (w/v), about 0.1% (w/v) to about 15% (w/v), about 0.1% (w/v) to about 20% (w/v), about 5% (w/v) to about 20% (w/v), about 10% (w/v) to about 20% (w/v), about 15% (w/v) to about 20% (w/v). The surfactant may be present at a concentration of about 0.1% (w/v), about 0.5% (w/v), about 1% (w/v) about 1.5% (w/v), about 2% (w/v), about 2.5% (w/v), about 3% (w/v), about 4% (w/v), about 5% (w/v), about 6% (w/v), about 7% (w/v), about 8% (w/v), about 9% (w/v), about 10% (w/v), about 11% (w/v), about 12% (w/v), about 13% (w/v), about 14% (w/v), about 15% (w/v), about 16% (w/v), about 17% (w/v), about 18% (w/v), about 19% (w/v), or about 20% (w/v). In exemplary embodiments, the surfactant may be present at a concentration from about 0.5% (w/v) to about 20% (w/v), about 0.5% (w/v) to about 5% (w/v), about 0.1% (w/v) to about 1.5% (w/v), or less than about 2% (w/v).

The composition of the present disclosure may have a pH from about 6.5 to about 11.5. For example, the composition may have a pH from about 6.5 to about 7.5, about 6.5 to about 8.5, about 6.5 to about 9.5, about 6.5 to about 10.5, about 6.5 to about 11.5, about 7.5 to about 11.5, about 8.5 to about 11.5, about 9.5 to about 11.5, about 10.5 to about 11.5, about 6.5 to about 8, about 9.5 to about 11.5, about 9 to about 11, or about 7 to about 10. The composition may have a pH of about 6.5, 7.0, 7.5, 8.0, 8.5, 9.0, 9.5, 10.0, 10.5, 11.0, or about 11.5. In some exemplary embodiments, the composition may have a pH higher than physiological pH.

In one version, the composition may be initially at a first pH relatively closer to physiological pH, and then the pH may be increased to a pH of 8.5 or higher. Without wishing to be bound by theory, it is believed that the use of two distinct pH ranges can, in some aspects, allow a thrombolytic agent to be effective over a time period sufficiently long to act effectively against clots while at a pH relatively closer to physiological pH, while the EDTA can be more effective in its antimicrobial and/or anti-biofilm functions at the higher pH range, thereby allowing both compounds to have relatively optimum performance.

In another embodiment, antiseptic compositions disclosed herein consisting of, consisting essentially of, or comprising one or more sodium salt(s) of EDTA at a pH greater than physiological pH are provided as sanitizing solutions for medical devices such as dentures and other dental and/or orthodontic and/or periodontal devices, for contact lenses and other optical devices, for medical and veterinary instruments, devices, and the like, and as sanitizing solutions for sanitizing surfaces and objects. In some embodiments, the pH is greater than 9.5, 9.5-11.5, or 9.5-10.5. Methods of sanitizing such devices are also provided, comprising contacting a device with antiseptic compositions disclosed herein. In general, antiseptic compositions disclosed herein may be used as soaking solutions for dental, orthodontic, and periodontal devices, including toothbrushes, and are also used as soaking solutions for contact lenses and other optical devices, and well as medical and veterinary instruments, devices, and the like. For these applications, antiseptic compositions disclosed herein are generally formulated as solutions or are provided in a dry form that forms a solution upon the introduction of a suitable solvent.

In yet another embodiment, antiseptic compositions disclosed herein are formulated for use in solutions, gels, creams, and other preparations designed for topical use as antiseptic agents, wipes, antibacterial treatments, and the like. Antiseptic compositions disclosed herein may also be used as antibacterial agents connected with bandages, dressings, wound healing agents and devices, sprays, and the like.

In still another embodiment, antiseptic compositions disclosed herein are used in industrial settings such as water storage and distribution systems, water purification, humidification, and dehumidification devices, and food preparation, handling, and packaging settings to inhibit, reduce or substantially eliminate microbial populations in both planktonic and sessile forms, as well as many fungal, amoebic, and planktonic populations. Industrial equipment and surfaces may be contacted or flushed with, or soaked in antiseptic compositions disclosed herein. Time-release antiseptic composition formulations may also provide treatment over time, particularly in locations that are difficult to access frequently.

Medications are often administered through luer connections, needleless connectors, and needle access ports. Connection sites need to be sterilized, which is often done with an antiseptic wipe. End-site connectors, catheter hubs, needle access ports, or needless connectors are often cleaned using an antiseptic towelette that comes in a small foil packet and is commonly used throughout hospitals, clinics, and home healthcare. The towelette may be a small, folded sheet of fibrous, nonwoven material that absorbs an amount of isopropyl alcohol. Alcohol, while effective, is not as effective in destroying biofilms as a combination of tetrasodium EDTA, ethanol, and chlorhexidine.

In various aspects, two or more types of antiseptic towelettes, such as towelettes with differing concentrations of agents in the wetting solution, may be provided that can be used for various tasks or at various times. In other embodiments of various methods, kits, product combinations, and the like for peripheral line disinfection, two or more solutions may be provided such as a catheter lock solution, a flush solution, a wet wipe wetting solution, a cleansing liquid, and the like, all of which may have different ingredients, different concentrations of identical or similar components, different pH values, and so forth

Without wishing to be limited by theory, Applicant proposes that any of several factors can result in a need to provide more than one type of towelette or solution. One such factor is the nature of the task to be performed. In some cases, different disinfecting tasks will require different disinfectant compositions. For example, the cleansing of a catheter needle or catheter tube before inserting the catheter may require a particular mix of antimicrobial agents, including agents that may be biocompatible in the patient's bloodstream, while disinfecting ports, caps, and hubs of a catheter may require a different mix, as is true of the task of disinfecting hands of caregivers or physicians where a low level of EDTA, even a substantially EDTA-free solution, may be sufficient, while cleaning the skin of a patient may require yet another composition. Further, using a towelette to clean a patient's skin at a catheter site before insertion may require a different mix (e.g., higher antimicrobial levels) than is ideal for subsequent maintenance of the site of catheter surfaces, or for a catheter lock or flush. Similar principles may apply to many disinfecting tasks for other surfaces and systems.

In addition to different compositions for different tasks, different substrates may be required, such as nonwoven or woven materials that vary in basis weight, texture, porosity, absorbency, strength, embossing, and amount of wetting solution added. For example, a relatively coarse and stronger substrate may help scrub a patient's skin before inserting a catheter needle, while a smoother, finer substrate may be most useful for cleaning a needle, port, or catheter line.

Another reason for using two or more types of disinfectant wipes, towelettes, or solutions is the diversity of biofilms and biofilm structures, including the different stages involved in biofilm formation over time. It is proposed that the change in biofilms over time may require different approaches for optimum treatment, including different concentrations of agents, to limit growth. For example, in a biological system with the liquid present, planktonic bacteria may undergo a stage of reversible attachment to a solid surface and monolayer formation, followed by irreversible attachment and microcolony formation, followed by maturation of the biofilm and then dispersal of bacteria back into the system. There may be a need for differing agents or concentrations of agents at each of these stages. For example, using combinations of EDTA and chlorhexidine, for some environments and some types of bacteria, it may be that a low concentration of EDTA initially may be suitable to reduce the ability of the bacteria to attach to the surface. Over time, however, some will attach and begin forming a biofilm. Once that is established, it may be that EDTA is no longer as effective and that what is needed is a higher concentration of chlorhexidine and/or the addition of taurolidine to prevent dispersal and kill planktonic bacteria released from the biofilm.

In some cases, though, it may be that the ability of EDTA to extract iron and calcium from the biofilm makes it particularly effective against, say, monolayer or early stages of maturation, such that an effective treatment strategy when there are indications of microcolony formation might involve using elevated EDTA levels for a period of time, optionally coupled with various levels of taurolidine or other agents described herein. Thus, while the catheter lock solution may be effectively adjusted over time—which may be replacing a first lock solution with a second lock solution having different concentrations or differing compounds—there may also be a need to adjust the supporting use of external products to maintain sterile conditions within or on the catheter and reduce the presence of pathogens on the skin near the catheter site. Such adjustment may include selecting a towelette type or solution from two or more towelette types or solutions having differing concentrations in one or more of the components in the solution, which may be a wetting solution. Such adjustment may also include applying a distinct catheter lock solution or flush solution.

For example, the concentration of EDTA and/or taurolidine in a first towelette type or solution may be substantially different than the EDTA and/or taurolidine concentration in the second towelette type or solution, such that concentration difference is at least 0.5% by weight (e.g., such as when one type has a concentration of 1.5% EDTA in the solution, which may be a wetting solution, and another type has a concentration of, say, 2.0% or 1.0% EDTA or taurolidine), or at least 1%, 1.5%, 2%, 2.5%, or 3% in concentration. Similar concentration ranges may apply to the chlorhexidine or other components of the wetting solution. In some versions, the one type of towelette or solution (e.g., a towelette solution, lock solution, flush solution, etc.) is substantially free of one ingredient (e.g., EDTA, chlorhexidine, taurolidine, ethanol, etc.) present in another type of towelette or solution. For example, the first type of towelette or first type of solution may have a solution, which may be a wetting solution, with 1% to 7% EDTA and from 0.5% to 3.5% chlorhexidine and less than 1% taurolidine such as 0.5% or substantially 0% taurolidine. In comparison, a second type of towelette or second type of solution may have a solution, which may be a wetting solution, having from 1% to 7% EDTA and from 0.5% to 3.5% chlorhexidine but at least 1% taurolidine such as 1% to 5%, 1% to 3%, or 2% to 7% taurolidine. Tetrasodium EDTA as used herein may comprise one or more, or all of, di-sodium, tri-sodium, and tetra-sodium EDTA. A tetrasodium EDTA concentration may be a combined di-sodium, tri-sodium and/or tetra-sodium EDTA concentration.

Another reason for employing two or more types of towelettes or solutions is the transient nature of bacteria populations in health care settings. A microbiome of bacteria resides on the skin, and the makeup of bacteria can vary from place to place and over time. The dominant species of bacteria or the distribution of bacterial species that may be present on a catheter surface, in or near a wound site, or on other surfaces or environments may change over time, in part at least due to the presence of antimicrobial agents such as those provided on the first type of towelette. As susceptible species decline and more resistant species rise in count or predominance over time (this can occur even within a single biofilm, where a variety of species may interact and some may rise or decline in prominence over time), there may be a need to apply a different kind of antimicrobial mix via the second type of towelette to more effectively control bacteria. This rise of a more resistant species can be true of bacteria on or in catheter hubs, various catheter lines and other parts of catheter systems, wound sites, etc., as well as on other surfaces such as fabrics or clothing (where biofilms such as dry biofilms may still be established), IV bags and equipment, door handles, electronic equipment, etc. Towelettes or other means of providing antimicrobial solutions may benefit from having two or more types that can be used periodically to reduce the risk of microbes resistant to one type of towelette rising in population and presenting a risk to a patient.

Yet another reason to consider multiple types of towelettes or solutions or antimicrobial treatments is the change in a patient and the patient's physiological that may occur over time. This change in the patient can be due to recovery from surgery, progression of a disease or other ailment, changes in diet, medication, the humidity of the room, etc., all of which may affect the microbes associated with a patient both internally and externally (including the skin microbiome, respiratory microbes, fingernail microbes, digestive tract microbes, urinary microbes, blood microbes, etc.). Such changes may require changes in the antimicrobial treatments applied to the skin, catheter hub, or in or on an IV line, and other places to reduce the risk of biofilm formation or microbial flare-ups, as well as to the catheter lock solution, etc. To cope with such changes, it may be useful, for example, to periodically apply a towelette or solution that has one or more components at a high concentration that might be too strong or costly for frequent use but suitable for brief intermittent use, such as the first type of wipe or solution with chlorhexidine at a concentration of 1% and EDTA at a concentration of from 2% to 4%, and a second towelette or solution with chlorhexidine at a concentration of 4% or greater such as from 4% to 8% and EDTA at a concentration of from 2% to 4%, wherein the first towelette or solution is used most frequently. In contrast, the second towelette or solution is only used once every day or once every period of, say, 3 or more hours, or 1 in 5 times or 1 in 3 times of towelette application, thereby giving an occasional spike in antimicrobial dosage. Alternatively, the second towelette or solution may comprise a different mix of antimicrobial components, such as adding taurolidine that was not present in the first towelette or solution. Regardless of the cause of changing needs in coping with microbial risks, the availability of two or more types of towelettes or solutions can support improved routines for fighting bacteria and give caregivers and medical workers additional tools to develop improved care regimens.

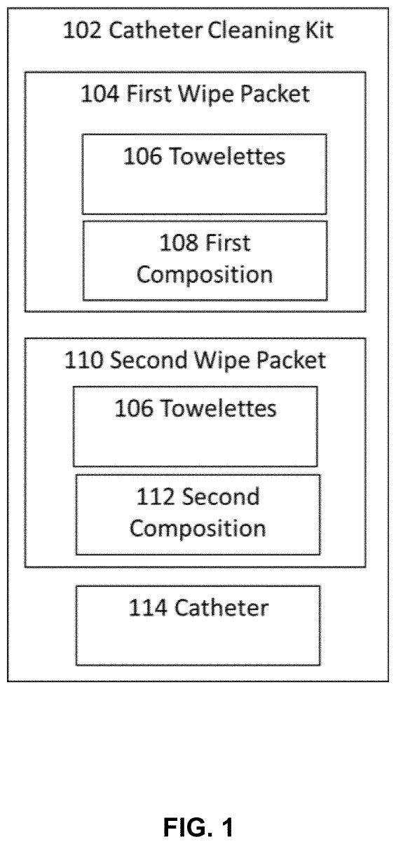

FIG. 1 illustrates a catheter cleaning kit 102, which may contain one or more wipe packets 104. Each wipe packet 104 may be a foil pouch that contains one or more towelettes 106. The towelettes 106 may be a small, folded sheet of fibrous, nonwoven material into which an amount of a first composition 108 has been absorbed. The towelettes 106 may exude the first composition 108 and may therefore be used to apply that first composition 108 to one or more surfaces. The first composition 108 may be a composition and solutions comprising about 1-15% (w/v) tetra-sodium EDTA, ethanol, and chlorhexidine, wherein the composition has a pH of at least 9.5. It may be biocompatible in a patient's bloodstream. In an area of concern such as on a catheter hub or near a catheter site or other surface, the towelettes 106 from the first type of towelette may be required initially to reduce the threat of infection. However, as biofilm develops, or as the distribution or count of microbes varies over time, or as the physiology of the patient changes, a need may arise for yet another type of towelette, as further discussed below.

In one embodiment, the first composition 108 may contain about 1-15% (w/v) tetra-sodium EDTA and at least 1% taurolidine, wherein the composition has a pH of between 6.5 and 7.5 and may be biocompatible with a patient's bloodstream.

Some versions may include a second wipe packet 110 with one or more towelettes 106 containing a second composition 112. The second composition 112 may be one of the various application-specific disinfecting compositions. In one embodiment, the second composition 112 contains about 1-15% (w/v) tetra-sodium EDTA, wherein the composition has a pH of between 6.5 and 7.5 and may be biocompatible in a patient's bloodstream. This second wipe packet 110 with the second type of towelette 106 having a second composition 112 may differ from the previously described first composition 108 in terms of overall concentration, the addition relative to the second composition of one or more components such as taurolidine, the concentration of one or more components, the absence of one or more components, etc. The second composition 112 may, for example, provide a stronger potency for one or more antimicrobial agents needed to undermine an emerging biofilm that may be detected or that may tend to form after a certain period of time has elapsed, or it may be needed to cope with expected or detected changes in microbial distribution or count, in biofilm formation, in biofilm stage and activity, in patient physiology due to medication or other factors, in the type of bacteria that are becoming more populous, etc. The change in the composition may be designed to kill certain species, to promote the presence of healthy bacteria on the skin to counter the rise of pathogens, to prevent adhesion of microbes to a surface, to disrupt the protective matrix of biofilms generally formed from extracellular polymeric substances (EPS), to prevent the release of active bacteria from a biofilm, to more effectively kills planktonic bacteria, etc.

In some cases, different disinfecting tasks will require different disinfectant compositions. For example, first composition 108 with a relatively high concentration of chlorhexidine in an alcohol solution may be useful for preparing the skin of a patient for catheter insertion. In contrast, a second composition with a lower concentration of chlorhexidine and/or a higher concentration of EDTA may be useful in later cleansing of catheter surfaces and sites in order to reduce the risk of biofilm formation.

Embodiments may include a catheter 114 or other medical devices that require disinfecting the patient and/or the device.

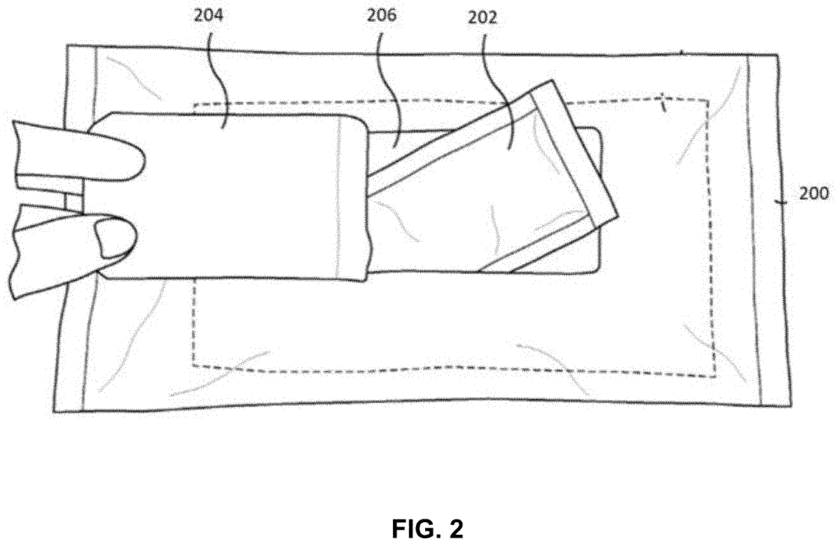

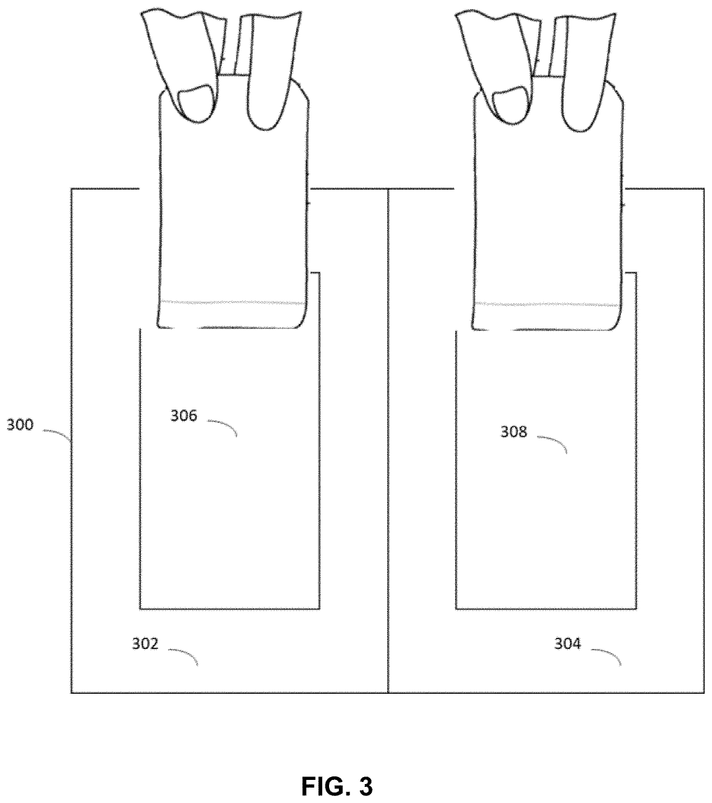

FIG. 2 illustrates a wipe dispenser 200, which may be a flexible sealed container containing a plurality of disinfecting wipes 202. These wipes may be impregnated with a composition which may be a composition and solutions comprising about 1-15% (w/v) tetra-sodium EDTA, ethanol, and chlorhexidine, wherein the composition has a pH of at least 9.5 and which, in some aspects, may be biocompatible in a patient's bloodstream. The composition may be a first composition intended for use in certain tasks, stages of treatment, physiological conditions, or in response to the absence or presence of biofilm formation or maturation indicators. It may be used alternately with or in contrast to a second composition (not shown) provided in another type of disinfecting wipes (not shown here) such as those of FIG. 3 below based on the tasks being carried out or in response to changes in microbes, biofilm stage, patient physiology, bacteria type encountered, etc. The disinfecting wipes 202 may be retrieved through an opening 206 in the wipe dispenser 200 that is exposed when the adhesive label 204 is peeled back. The adhesive label 204 may be re-attached to the wipe dispenser 200 once the disinfecting wipe 202 has been removed so as to prevent the evaporation of the disinfecting composition. The wipe dispenser 200 may be made of a rigid outer material in some embodiments. The wipe dispenser 200 may be affixed to a wall or other surface.

FIG. 3 illustrates a two-composition wipe dispenser 300. The wipe dispenser may be a flexible or rigid plastic container with two compartments. A first compartment 302 contains disinfecting wipes that may be impregnated with a first composition which may be a composition and solutions comprising about 1-15% (w/v) tetra-sodium EDTA, ethanol, and chlorhexidine, wherein the composition has a pH of at least 9.5. It may be biocompatible in a patient's bloodstream. Relative to the disinfecting wipes 202 of FIG. 2, the disinfecting wipes here may represent a second type of disinfecting wipe having a second composition that may be suitable for different tasks or for responding to different indications of biofilm formation or biofilm maturation stages, different states of patient physiology such as different states of catheter wound health or different bioindicators (biomarkers for bacteria species, measurement results for temperature, medication, blood pressure, etc.), etc. The second composition may have one or more components relative to the first composition in the first type of towelettes wherein at least one antimicrobial component is at a substantially different concentration, differing by, say, at least 25%, at least 50%, at least 75%, or at least 100% in concentration (higher or lower). In another aspect, at least two components have substantially different concentrations.

A second compartment 304 may contain disinfecting wipes that may be impregnated with a second composition which may be a composition and solutions comprising about 1-15% (w/v) tetra-sodium EDTA, ethanol, and chlorhexidine, wherein the composition has a pH of at least 9.5. It may be biocompatible in a patient's bloodstream. The composition may be adapted for specific objectives such as maintaining a sterile area with a relatively low concentration of antimicrobials or cleansing a potentially infected area with a relatively high concentration of active ingredients. Alternatively, it may be adapted for cleaning a surface that will have contact with the bloodstream of a patient and thus may be provided with a more biocompatible formulation, or it may be adapted for fighting a biofilm that has become established on a solid surface and thus may, for example, require a relatively higher concentration of EDTA and other agents such as chlorhexidine, taurolidine, or both. In one aspect, the composition may be used to undermine an established biofilm with about 2-6% (w/v) tetrasodium EDTA, such as about 3%-(w/v) tetrasodium EDTA, about 10-60% (w/v) ethanol, such as about 20-25% (w/v) ethanol, and about 0.5-6% (w/v) chlorhexidine HCl, such as about 1-2% (w/v) chlorhexidine, optionally with about 0.5-2% (w/v) taurolidine, and optionally at a pH of 9 to 11.5, such as a pH of about 9.5.

Biofilms associated with implantable medical devices and wounds are clinically relevant, often requiring repeated use of antibiotics without success. The use of non-antibiotic antimicrobial and antibiofilm solutions is in line with antimicrobial stewardship. The antimicrobial efficacy of tetrasodium EDTA, ethanol, and chlorhexidine hydrochloride (HCl) in combination has been found effective against clinically relevant planktonic and biofilm cells of bacterial and fungal pathogens.

Biofilms on catheters are responsible for catheter-related bloodstream infections (CRBSIs), which cause significant mortality and morbidity. Antimicrobial catheter-lock solutions may salvage precious catheters by eradicating biofilms. Staphylococcus epidermidis and Candida albicans are frequently isolated organisms in CRBSIs.

Against planktonic cells, the combination of tetrasodium EDTA with ethanol or chlorhexidine HCl resulted in synergistic effects. Against mature biofilms, all combinations were synergistic. An optimized concentration of antimicrobials is used to achieve rapid eradication of pre-formed biofilms. A triple combination of about 3% (w/v) tetrasodium EDTA, about 20% (w/v) ethanol, and chlorhexidine HCl has been shown to completely eradicate 48-h-old biofilms of all test strains within 2 hours.

Biofilms can be particularly dangerous when they grow on medical devices inserted into the body. Specifically, biofilms present on intravascular catheters (IVCs) or central lines represent the most common cause of hospital-acquired septicemias: catheter-related bloodstream infections (CRBSIs).5 IVCs, which are often left in place for several weeks, provide a means to draw blood and administer medications and nutrition and furnish bacteria with an easily traversable superhighway leading directly into the bloodstream. And when microorganisms introduced from the skin of the patient at the catheter insertion site, from a contaminated catheter hub, or from hematogenous seeding of the device can attach to the device's external surfaces and lumen (internal surfaces), infection becomes likely. Studies have shown that biofilms may form within three days after catheter insertion and tend to form on the external surface of catheters in place for less than 10 days; however, with increasing catheter duration (greater than or equal to 30 days), biofilms tend to form in the catheter lumen.

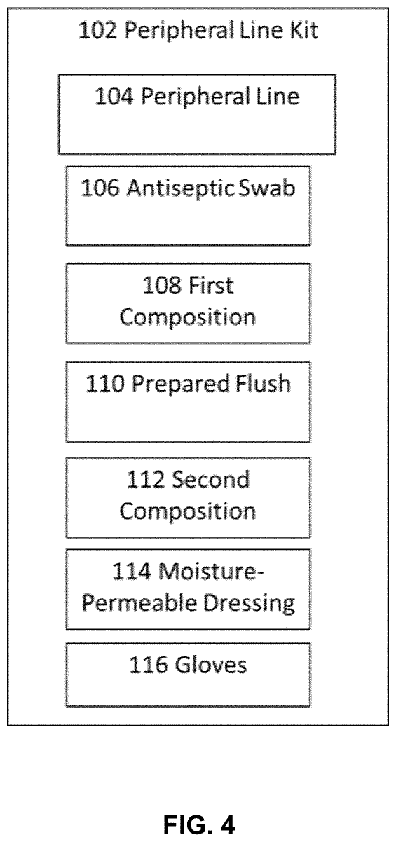

FIG. 4 illustrates a peripheral line placement kit 402, which may contain an IV Line 404. The IV Line 404 may be a hollow, plastic, or polyurethane, tube-shaped catheter that connects to a larger hub (not shown) outside the skin. The IV Line 404 may be coupled to a beveled needle with a transparent chamber to allow the user to identify when the needle begins receiving blood from the vein. The hub may be color-coded to communicate the gauge of the needle. Needle gauge may vary depending on patient characteristics. The length of the catheter can vary between different manufacturers. Removing the needle from the external hub of the line reveals a standard Luer-taper connector to which a phlebotomy adapter, a needle-free injection bung, or an IV fluid administration set can be attached. The hub may also have a side port with a removable cap, allowing drug administration without disconnecting other ongoing IV fluids or medications.

The peripheral line placement kit 402 may also include some number of antiseptic swabs 406. The antiseptic swab 406 may also be in the form of a sponge, wipe, or towelette. The antiseptic swab 406 may contain a first composition 408. The first composition 408 may be a composition and solutions derived from the combination of tri-sodium and tetra-sodium EDTA, ethanol, and chlorhexidine at a combined tri-sodium and tetra-sodium EDTA concentration of at least 1.0% (w/v) and less than 15% (w/v), and wherein the composition has a pH of at least 9.5 and is biocompatible in a patient's bloodstream. Such a solution can effectively control bacteria on surfaces such as a catheter hub, ports, tubes, etc., and the skin of a patient or the hands of caregivers, though the specific concentration of ingredients may be adapted to the specific task or use. For example, a relatively lower EDTA concentration or lower taurolidine concentration may be suitable for handwashing compared to disinfecting a catheter tube or hub.

The peripheral line placement kit 402 may also contain a prepared flush 410. The prepared flush 410 or a prepared bag of IV fluid with an administration may be used to confirm adequate flow, observing for the absence of swelling or edema around the insertion site. The prepared flush 410 may use sterile saline. In one embodiment, the prepared flush 410 may contain a second composition 412 may contain about 1-15% (w/v) tetra-sodium EDTA, and at least about 1% (w/v) taurolidine, wherein the composition has a pH of between 6.5 and 7.5 and is biocompatible in a patient's bloodstream. The flush 410 is adapted for different tasks or conditions than the first composition 408 and may have a relatively higher concentration of at least one ingredient relative to the first composition 408 or any other solution provided in the kit. For example, the flush 410 may have a component such as EDTA, taurolidine, chlorhexidine, etc. whose concentration is at least 20% higher, 50% higher, or 100% higher than the concentration of the same component in the first composition 408, or on an absolute weight percentage basis may have a concentration at least 0.5%, 1%, 2%, 5% or 8% greater than the concentration of the corresponding component in the first composition 408 or other solution provided in the kit. For example, the flush 410 may have about 2% (w/v) taurolidine while the first composition 408 is substantially free of taurolidine, or the flush 410 may about 10-30% (w/v) ethanol, while the first composition may have about 40% (w/v) or more ethanol. Because the purpose of the flush 410 may be primarily to prevent occlusion of the catheter with periodic flushing of small amounts of fluid that enter the bloodstream, urine, or other physiological material (e.g., 3 ml or 5 ml flushes), the flush 410 may be adapted to be biocompatible with the physiology of the patient, while the solution used with a swab, wipe, or surface cleaning product need not be.

More than one type of flush 410 or catheter lock solution may be provided to cope with ongoing changes in bacteria, biofilm stage, patient physiology, condition, etc. Thus, there may be a first and second flush 410 or first and second catheter lock solution (not shown) having varying concentrations of antimicrobial or other ingredients to provide options to cope with changes over time or for different tasks. The peripheral line placement kit 402 may contain a moisture permeable dressing 416 and may contain gloves 418.

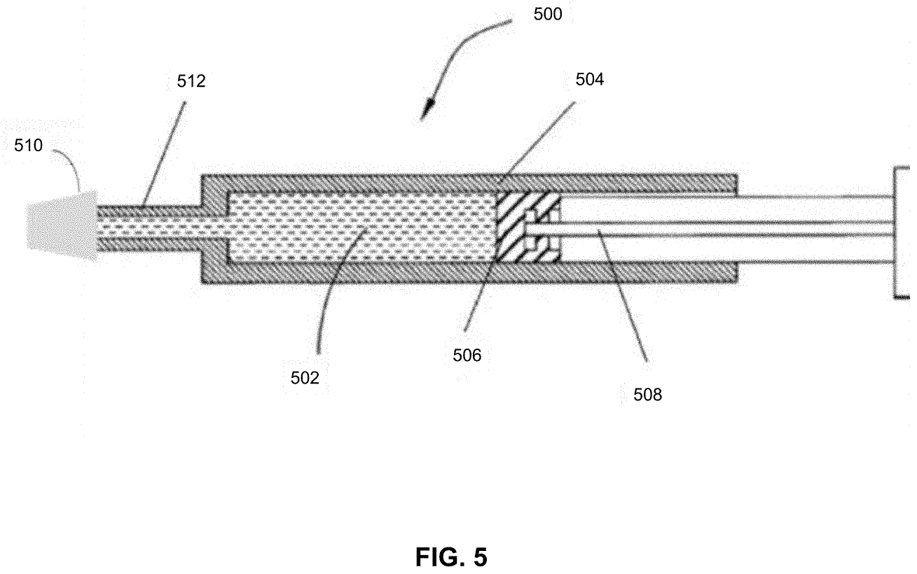

FIG. 5 illustrates a pre-filled syringe 500 filled with a lock solution 502. The syringe 500 includes a barrel 504, a seal 506, plunger 508, cap 510, and luer lock 512. The plunger 508 has a shaft that connects to the seal 506. Pressure is applied through this shaft to the seal, where it presses on the lock solution 502 in the barrel 504. The output of the syringe 500 is formed into a luer lock 512. The luer lock 512 may be a standardized screw fitting for attached fitting, such as luer access devices (LAD), that will not blow off when the liquid is under pressure. In other embodiments, a luer lock 512 may be a luer taper. A luer taper allows for leak-free connections to be made between standardized small-scale fluid fittings. Luer taper fittings that are not luer lock fittings allow for luer slip fittings held in place by friction when there is no thread. The lock solution 502 may differ in the concentration of one or more antimicrobial agents for reasons explained above and need not have the same concentrations as the flush solution 410 of FIG. 4.

Table 1 illustrates the matrix of compositional ranges appropriate for one or more applications disclosed herein.

TABLE 1

| | First composition | Second composition |
|---|---|---|
| | (e.g., towelette, | (e.g., towlette, |
| | solution, or peripheral | solution, or PICC |
| Component | line concentration) | line concentration) |
| Carrier | 76% | 79% |
| Tetrasodium EDTA | 3% | 10% |
| Ethanol | 20% | 10% |
| Chlorohexidine | <1% | <1% |
| pH | >9.5 | >9.5 |

The first composition may be used to disinfect a peripheral line. The second composition may be used to disinfect a PICC line.

Taurolidine may be present in catheter locks, flush solutions, swab solutions, wipe solutions, or any other products in the kit at concentrations such as from about 0.3% to 4% (w/v), 0.5% to 3%, 0.5% to 2.5%, 1% to 5%, 1% to 2.5%, etc., or less than 3%, less than 2%, less than 1%, less than 0.5%, less than 0.3%, less than 0.1%, or substantially free of taurolidine. Chlorhexidine may be present at concentrations such as from about 0.2% to 4% (w/v), 0.3% to 4%, 0.5% to 6%, 0.5% to 4%, 0.5% to 3%, 1% to 4%, etc., or less than 3%, less than 2%, less than 1%, less than 0.5%, less than 0.3%, less than 0.1%, or substantially free of chlorhexidine. Ethanol may be present from about 0% to 70% (w/v), such as from 10% to 70%, 10% to 40%, 10% to 25%, 20% to 70%, 30% to 70%, 20% to 60%, etc., or less than 10%, 5%, 3%, 2%, 1%, or substantially ethanol free. EDTA (collectively for all ionic forms or for any one, two, or three ionic forms together) may range from about 1% to 15% (w/v), 1% to 12%, 1% to 10%, 1% to 8%, 1% to 5%, 0.5% to 4%, 0.5% to 3%, etc., or less than 1%, less than 0.5%, or substantially free of EDTA. Percentages are weight percentages unless otherwise indicated.

### EXAMPLES

### Example 1

An organisms and culture composition included (1) test pathogens of a single isolate from the species Stenotrophomonas maltophilia (ON17), Proteus mirabilis (ON153), Pseudomonas aeruginosa (SK1), and Serratia marcescens (SI<2) as well as (2) two isolates from the species Staphylococcus epidermidis (ON170 and SK9), S. aureus (ON89 and ON184), E. coli (ON29 and SK2) and Candida albicans (ON47 and SK4b).

An antimicrobials composition included a KITELOCK™ 4% Sterile Catheter Lock Solution (40 mg/mL tetrasodium EDTA) by SterileCare Inc., which is distinct from standard ‘disodium’ EDTA that is prepared at near-neutral pH; the pH of the KiteLock™ solution is near 11. The high pH does not kill micro-organisms directly but changes EDTA to the tetrasodium form, which has increased microbial killing effects. Chlorhexidine HCl was purchased from Sigma-Aldrich (product #C8527-5G). The antimicrobials composition (1 mg/mL) was made by dissolving the appropriate amount of chlorhexidine HCl powder in distilled water heated to 50° C., allowing the solution to cool and passing it through a 0.22 μm filter.

An assay was made using a minimum inhibitory concentration (MIC) and minimum bactericidal concentration (MBC) and minimum fungicidal concentration (MFC) determination. The MIC was determined by the micro broth dilution method in 96-well plates. Serial two-fold dilutions of tetrasodium EDTA (from 2% to 0.015%), ethanol (from 50% to 0.1%) and chlorhexidine HCl (from 100 μg/mL to 0.025 μg/mL) were prepared in MH broth with a final volume of 90 μL per well. A 10 μL containing 1×10⁵ bacterial cells or 2×10³ fungal cells were added to each well. The inoculated plates were covered with a lid, sealed with Parafilm, and incubated for 24 h at 37° C. with slight rocking on a tilting platform shaker. After incubation, the optical density at 600 nm (OD 600) of the cultures in each well was measured using an xMark™ Microplate Absorbance Spectrophotometer (Bio-Rad). The MIC was defined as the lowest concentration of antimicrobial compound at which the culture OD600 values were similar to uninoculated control wells. MBCs and MFCs were determined by transferring 100 μL from each well with no apparent growth onto appropriate agar plates, followed by incubation for 24 h at 37° C.

A fourth composition and solution included tetrasodium EDTA with ethanol or chlorhexidine HCl. This fourth composition and solution were created using checkerboard titration methods using micro broth dilution in 96-well microtiter plates. The concentrations of antimicrobials used were based on previously determined MIC values. Briefly, 200 μL, of two-fold dilutions of tetrasodium EDTA and ethanol or chlorhexidine HCl were prepared in MH or MH II broth with standardized cell suspension. The plate contained decreasing concentrations of tetrasodium EDTA (2%-0.015%) in columns 1-10 and decreasing concentrations of ethanol (50%-0.4%) or chlorhexidine HCl (50 μg/mL-0.0125 μg/mL) in rows A-H. Then, 10 μL of standardized cell suspension was added to each well. Microtiter plates were incubated at 37° C. for 24 h, and the results were analyzed. Each test was performed in duplicate and included a growth control without adding any antimicrobials.

A biofilm cultivation cell composition was provided. This fifth composition was created using an MBEC Assay® biofilm inoculator, consisting of a polystyrene lid with 96 downward-protruding pegs and a corresponding base used to grow biofilms. A standardized inoculum was diluted in an appropriate biofilm growth medium to achieve a viable cell count of 1.5×10⁶ CFU/mL of bacterial cells or 5×10⁵ CFU/mL of fungal cells. Then, 150 μL of this inoculum was transferred into each appropriate well, and the peg lids were inserted into the microtiter plates. The plates were sealed with Parafilm and were incubated at optimum temperature for 48 h with slight rocking for bacteria and shaking at 200 rpm for fungal strains. After incubation, the peg lid was removed from the base and rinsed twice with sterile phosphate-buffered saline (PBS) for 2 min to remove loosely attached non-sessile cells. Before the antimicrobial challenge, the pegs in column 1 (n=8) were considered the biofilm growth control; these pegs were removed from the lids, placed into 200 μL of recovery medium, and analyzed for starting biofilm cell numbers as described below. The rinsed pegs were placed into new 96-well plates containing two-fold dilutions of antimicrobials such as tetrasodium EDTA (4%-0.0125%), ethanol (100%-0.2%), and chlorhexidine HCl (100 μg/mL-0.4 μg/mL) in 200 μL of suitable biofilm growth medium per well and incubated at optimum temperature for 24 h. After the antimicrobial challenge, the pegs were rinsed twice with sterile PBS for 2 min and placed into a new 96-well plate containing 200 μL of recovery medium. The recovery plates were sealed with Parafilm, and biofilm cells were dislodged from the pegs by sonication for 30 min with a Branson 3510 bath sonicator. The biofilm cells in the recovery medium were serially diluted, and a drop dilution assay was performed to enumerate the viable cells. MBEC values were determined as the minimum concentration of antimicrobials that yielded a viable cell count at or lower than the 125 CFU/mL detection limit.

Determining the fractional biofilm eradication concentration (FBEC) index included the steps of (1) identifying synergistic antimicrobial effects of tetrasodium EDTA with either ethanol or chlorhexidine HCl on established biofilms, (2) using the ‘checkerboard dilution method’ where (3) pegs containing biofilms were treated with a combination of tetrasodium EDTA and ethanol or with tetrasodium EDTA and chlorhexidine HCl in 200 μL of two-fold dilutions inappropriate biofilm growth medium. This was followed by step (4) that included eight dilution steps of tetrasodium EDTA (4%-0.015%) either with ethanol (50%-0.4%) or chlorhexidine HCl (50 μg/mL-0.4 μg/mL) and where eight growth controls are analyzed for synergistic biofilm eradication. In step (4), microtiter plates are incubated at 37° C. for 24 h. then (6), after incubation, the bacterial and fungal cells were dislodged from the pegs into the recovery medium described above.

Three 10-μL aliquots, for a total of 30 μL from each well of recovery medium, were spotted on MH agar plates and incubated for 24 h at 37° C. The FBEC is the minimum concentration of antimicrobials in combination that completely inhibited bacterial or fungal growth on agar plates. The FBEC determination is a modification of the FICI.

Determining rapid biofilm eradication by tetrasodium EDTA, ethanol, and chlorhexidine HCl alone and in combination was performed. After biofilm formation, control pegs (n=6) were removed and analyzed to determine the starting biofilm cell numbers via the drop dilution method. The 48-h old biofilms on the pegs were exposed to different concentrations of test antimicrobials, dissolved in an appropriate growth medium, for two h to evaluate their efficacy alone and in combination. Antimicrobial solutions tested against each organism included each agent alone at the MBEC, double combinations at the FBEC, and triple combinations ranging from 5 to 20% ethanol, 2.5-5 μg/mL chlorhexidine HCl and 1-3% tetrasodium EDTA. Following treatment, pegs were washed twice with sterile PBS, and the biofilm cells were dislodged into recovery medium and enumerated as described above.

Antimicrobial activity of tetrasodium EDTA alone and in combination with either ethanol or chlorhexidine HCl against planktonic cells was determined. All three antimicrobials significantly inhibited the growth of all test organisms with MICs ranging from 0.063% to 2% for tetrasodium EDTA, 3.125%-12.5% for ethanol, and 0.1 μg/mL-50 μg/mL for chlorhexidine HCl. Synergy (FICI<0.5) was detected with the combination of tetrasodium EDTA with ethanol for all test Gram-positive and fungal strains, whereas partial synergy (0.5<FICI<1.0) was observed for all Gram-negative strains. The combination of tetrasodium EDTA with chlorhexidine HCl showed indifferent activity (1<FICI<4) against 4 of 12 test strains and synergistic or partially synergistic activity against the eight remaining strains (Tables 2-4 below).

TABLE 2
Minimum Inhibitory Concentration of Tetrasodium
EDTA, Ethanol, and Chlorhexidine HCl alone

| | MIC | | |
|---|---|---|---|
| | | | Chlorhexidine |
| | EDTA | Ethanol | HCl |
| Organism | (% w/v) | (% w/v) | (μg/ml) |
| Staphylococcus epidermidis | 0.063 | 3.125 | 0.1 |
| ON170 | | | |
| S. epidermidis SK9 | 0.063 | 6.25 | 0.1 |
| Staphylococcus aureus | 0.063 | 6.25 | 0.1 |
| ON89 | | | |
| MRSA ON184 | 0.063 | 6.25 | 0.2 |
| Stenotrophomonas maltophilia | 0.063 | 6.25 | 0.8 |
| ON17 | | | |
| Pseudomonas aeruginosa SK1 | 0.25 | 3.125 | 1.6 |
| Serratia marcescens SK2 | 2 | 6.25 | 12.5 |
| Proteus mirabilis ON153 | 1 | 6.25 | 50 |
| Escherichia coli ON29 | 0.5 | 12.5 | 0.4 |
| E. coli SK2 | 0.5 | 12.5 | 0.4 |
| Candida albicans SK4b | 1 | 6.25 | 1.6 |
| C. albicans ON47 | 1 | 6.25 | 1.6 |

TABLE 3
Fractional Inhibitory Concentration Index (FICI)
of Tetrasodium EDTA in Combination with Ethanol

| | EDTA | Ethanol | | |
|---|---|---|---|---|
| Organism | (% w/v) | (% w/v) | FICI | |
| Staphylococcus epidermidis | 0.015 | 0.4 | 0.36 | (S) |
| ON170 | | | | |
| S. epidermidis SK9 | 0.015 | 0.4 | 0.3 | (S) |
| Staphylococcus aureus | 0.015 | 0.4 | 0.3 | (S) |
| ON89 | | | | |
| MRSA ON184 | 0.015 | 0.4 | 0.3 | (S) |
| Stenotrophomonas maltophilia | 0.031 | 0.4 | 0.72 | (PS) |
| ON17 | | | | |
| Pseudomonas aeruginosa SK1 | 0.031 | 3.125 | 0.56 | (PS) |
| Serratia marcescens SK2 | 0.5 | 3.125 | 0.75 | (PS) |
| Proteus mirabilis ON153 | 0.5 | 1.6 | 0.75 | (PS) |
| Escherichia coli ON29 | 0.25 | 3.125 | 0.75 | (PS) |
| E. coli SK2 | 0.015 | 6.25 | 0.53 | (PS) |
| Candida albicans SK4b | 0.25 | 1.6 | 0.5 | (S) |
| C. albicans ON47 | 0.25 | 0.4 | 0.314 | (S) |

TABLE 4
Fractional Inhibitory Concentration Index (FICI) of Tetrasodium
EDTA in Combination with Chlorhexidine HCl

| | | Chlorhexidine | | |
|---|---|---|---|---|
| | EDTA | HCl | | |
| Organism | (% w/v) | (μg/ml) | FICI | |
| Staphylococcus epidermidis | 0.063 | 0.05 | 1.5 | (I) |
| ON170 | | | | |
| S. epidermidis SK9 | 0.015 | 0.025 | 0.5 | (S) |
| Staphylococcus aureus | 0.008 | 0.1 | 1.126 | (I) |
| ON89 | | | | |
| MRSA ON184 | 0.008 | 0.2 | 1.126 | (I) |
| Stenotrophomonas maltophilia | 0.015 | 0.2 | 0.5 | (S) |
| ON17 | | | | |
| Pseudomonas aeruginosa SK1 | 0.5 | 0.8 | 2.5 | (I) |
| Serratia marcescens SK2 | 0.063 | 1.5 | 0.16 | (S) |
| Proteus mirabilis ON153 | 0.063 | 0.8 | 0.08 | (S) |
| Escherichia coli ON29 | 0.008 | 0.2 | 0.516 | (PS) |
| E. coli SK2 | 0.008 | 0.1 | 0.266 | (S) |
| Candida albicans SK4b | 0.063 | 0.8 | 0.596 | (PS) |
| C. albicans ON47 | 0.125 | 0.8 | 0.658 | (PS) |

The three antimicrobial agents displayed broad-spectrum microbicidal activity against the 12 test organisms. MBC or MFC values of all test antimicrobials were equal to or higher than their respective MICs. The combination of tetrasodium EDTA with either ethanol or chlorhexidine HCl showed synergistic and partially synergistic activity against all the test strains except S. epidermidis ON170, which showed additive activity with an FMCI of 1.0. The nature of interaction found in FICI was not always the same as the FMCI. However, none of the tested tetrasodium EDTA, ethanol, or chlorhexidine HCl combinations showed antagonism concerning the FICI and FMCI values. These results are shown in Tables 5-7.

TABLE 5
Minimum Bactericidal Concentration (MBC) or
Minimum Fungicidal Concentration (MFC) of
Tetrasodium EDTA, Ethanol, and Chlorhexidine HCl alone.

| | MBC/MFC | | |
|---|---|---|---|
| | | | Chlorhexidine |
| | EDTA | Ethanol | HCl |
| Organism | (% w/v) | (% w/v) | (μg/mL) |
| Staphylococcus | 0.5 | 6.25 | 0.8 |
| epidermidis ON170 | | | |
| S. epidermidis SK9 | 0.5 | 12.5 | 0.8 |
| Staphylococcus aureus | 1 | 25 | 0.8 |
| ON89 | | | |
| MRSA ON184 | 2 | 25 | 1.6 |
| Stenotrophomonas | 1 | 6.25 | 3.125 |
| maltophilia ON17 | | | |
| Pseudomonas | 1 | 12.5 | 3.125 |
| aeruginosa SK1 | | | |
| Serratia | 2 | 12.5 | 25 |
| marcescens | | | |
| SK2 | | | |
| Proteus mirabilis | 2 | 12.5 | 50 |
| ON153 | | | |
| Escherichia coli ON29 | 1 | 25 | 0.8 |
| E. coli SK2 | 0.5 | 25 | 0.8 |
| Candida albicans SK4b | 1 | 6.25 | 3.125 |
| C. albicans ON47 | 1 | 6.25 | 3.125 |

TABLE 6
Fractional Microbicidal Concentration Index (FMCI) of
Tetrasodium EDTA in Combination with Ethanol

| Organism | EDTA (% w/v) | Ethanol (% w/v) | FMCI |
|---|---|---|---|
| Staphylococcus | 0.031 | 1.6 | 0.318 (S) |
| epidermidis ON170 | | | |
| S. epidermidis SK9 | 0.031 | 3.125 | 0.312 (S) |
| Staphylococcus aureus | 0.25 | 3.125 | 0.375 (S) |
| ON89 | | | |
| MRSA ON184 | 0.031 | 3.125 | 0.14 (S) |
| Stenotrophomonas | 0.125 | 1.6 | 0.381 (S) |
| maltophilia ON17 | | | |
| Pseudomonas | 0.031 | 6.25 | 0.531 (PS) |
| aeruginosa SK1 | | | |
| Serratia | 0.25 | 6.25 | 0.625 (PS) |
| marcescens | | | |
| SK2 | | | |
| Proteus mirabilis | 0.063 | 6.25 | 0.53 (PS) |
| ON153 | | | |
| Escherichia coli ON29 | 0.25 | 6.25 | 0.5 (S) |
| E. coli SK2 | 0.25 | 6.25 | 0.75 (PS) |
| Candida albicans SK4b | 0.25 | 3.125 | 0.75 (PS) |
| C. albicans ON47 | 0.25 | 3.125 | 0.625 (PS) |

TABLE 7
Fractional Microbicidal Concentration Index (FMCI) of Tetrasodium
EDTA in Combination with Chlorhexidine HCl

| | EDTA | Chlorhexidine HCl | |
|---|---|---|---|
| Organism | (% w/v) | (μg/mL) | FMCI |
| Staphylococcus | 0.25 | 0.4 | 1 (A) |
| epidermidis ON170 | | | |
| S. epidermidis SK9 | 0.008 | 0.4 | 0.516 (PS) |
| Staphylococcus aureus | 0.008 | 0.2 | 0.375 (S) |
| ON89 | | | |
| MRSA ON184 | 0.125 | 0.4 | 0.312 (S) |
| Stenotrophomonas | 0.031 | 0.4 | 0.16 (S) |
| maltophilia ON17 | | | |
| Pseudomonas | 0.031 | 1.6 | 0.543 (PS) |
| aeruginosa SK1 | | | |
| Serratia | 0.125 | 1.6 | 0.126 (S) |
| marcescens | | | |
| SK2 | | | |
| Proteus mirabilis | 0.125 | 1.6 | 0.09 (S) |
| ON153 | | | |
| Escherichia coli ON29 | 0.008 | 0.2 | 0.258 (S) |
| E. coli SK2 | 0.015 | 0.2 | 0.258 (S) |
| Candida albicans SK4b | 0.125 | 0.8 | 0.381 (S) |
| C. albicans ON47 | 0.031 | 1.6 | 0.51 (PS) |

Compositions of tetrasodium EDTA alone and in combination with either ethanol or chlorhexidine HCl against 48-h old, preformed biofilms may be created using a single antimicrobial agent, effective at eradicating preformed biofilms of test pathogens, with concentrations between 4% to 0.0125% of tetrasodium EDTA, 100%-0.2% of ethanol, and 100 μg/mL-0.8 μg/mL of chlorhexidine HCl. As per CLSI guidelines, the MBEC is defined as the minimum concentration of an antimicrobial that eradicates 99.9% of micro-organisms (i.e., 3-log reduction) in a biofilm state compared with their respective growth controls in similar conditions. All antimicrobials achieved >99.99% (i.e., 4-log reduction) killing of bacterial biofilm cells, whereas the starting biofilm cell numbers for C. albicans were not enough to achieve a clinically recommended standard of biofilm killing.

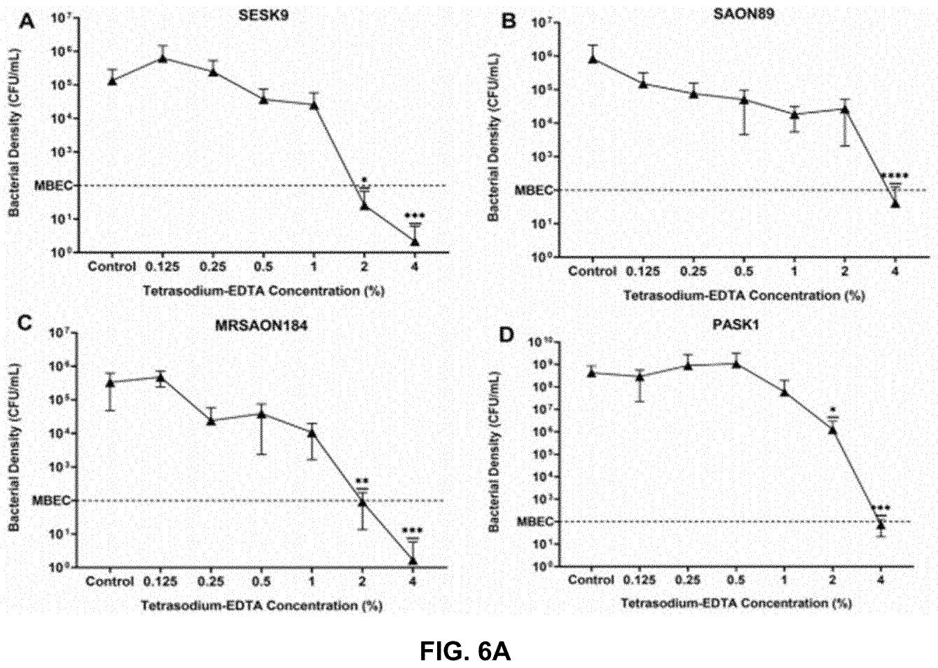

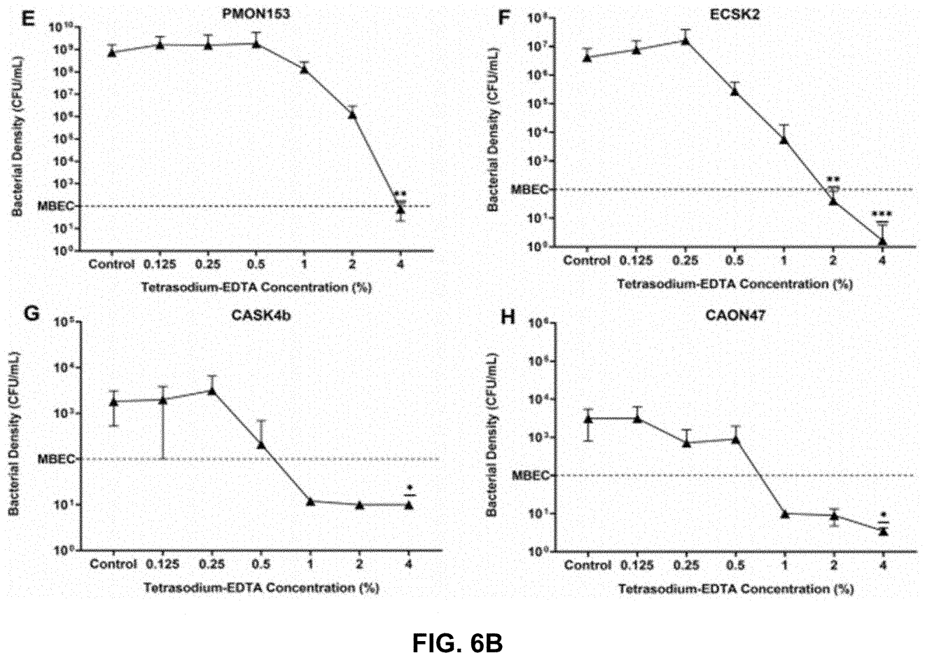

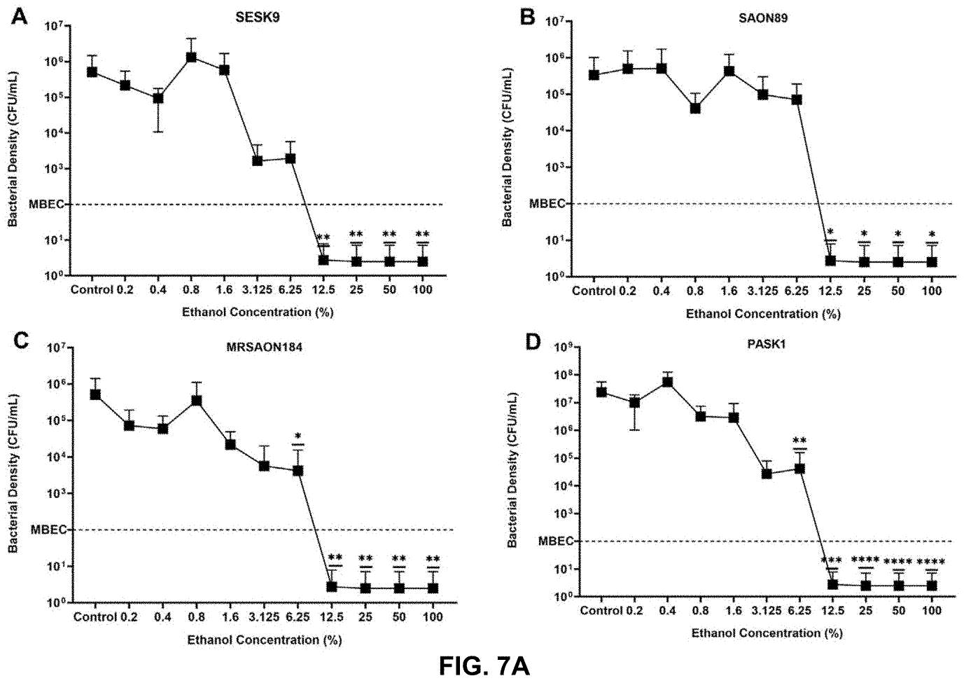

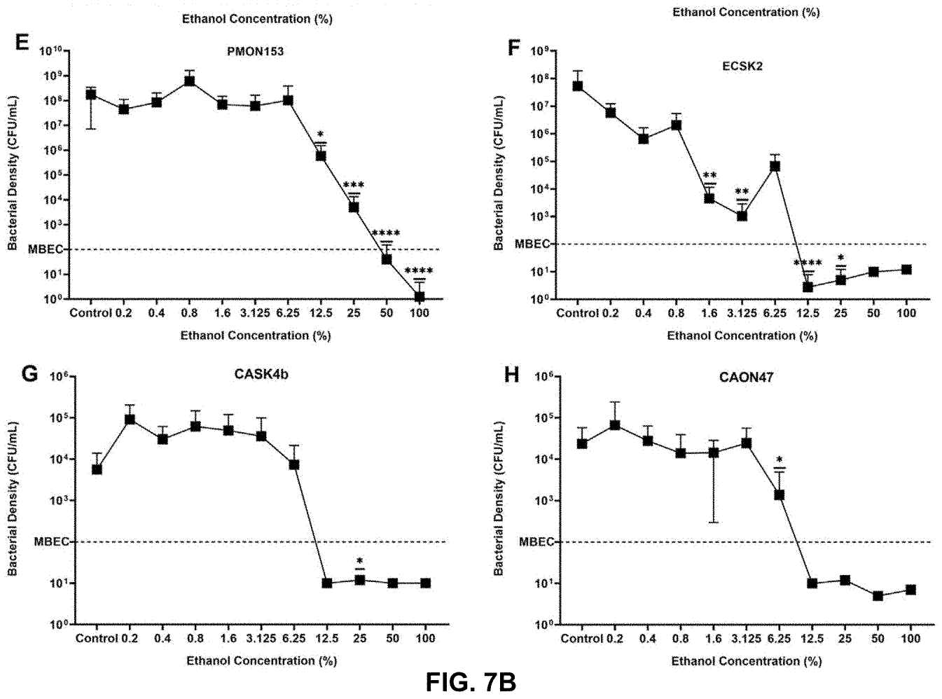

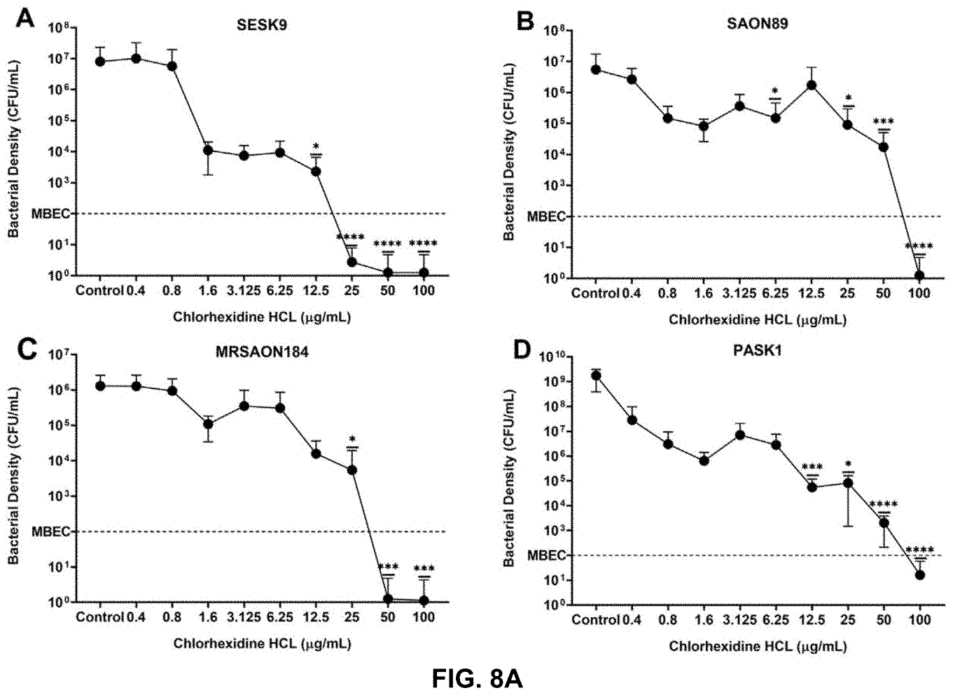

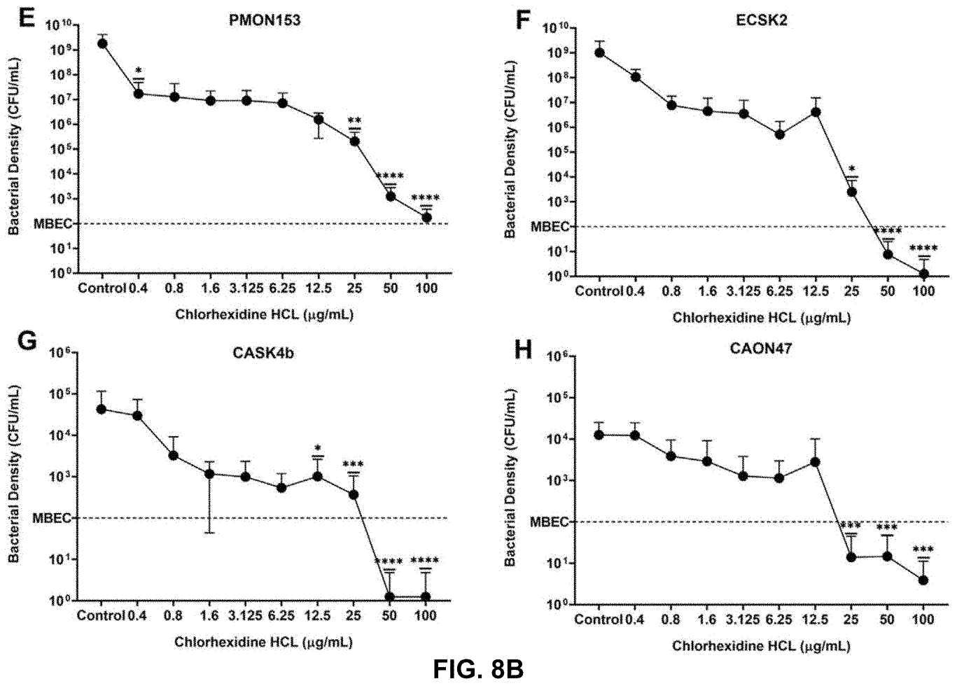

The MBEC of each antimicrobial agent against each test strain was established, and the data were plotted as the log reduction in the number of CFU (FIGS. 6A-8B). When tetrasodium EDTA was combined with either ethanol or chlorhexidine HCl, they exhibited a synergistic effect against all test strains in the study (Tables 8-10). According to the FBEC index, the concentration of tetrasodium EDTA in combination was decreased from ⅛- to 1/64-fold (with ethanol) and 1/16- to 1/64-fold (with chlorhexidine) in comparison with its original MBEC values. Also, the required concentrations dropped by ¼- to 1/16-fold for ethanol and ⅛- to 1/32-fold for chlorhexidine HCl when combined with tetrasodium EDTA (Tables 8-10).

TABLE 8
Minimum Biofilm Eradication Concentration (MBEC) of
Tetrasodium EDTA in Combination with Chlorhexidine HCl

| | MBEC | | |
|---|---|---|---|
| | | | Chlorhexidine |
| | EDTA | Ethanol | HCl |
| Organism | (% w/v) | (% w/v) | (μg/mL) |
| Staphylococcus | 2 | 12.5 | 25 |
| epidermidis SK9 | | | |
| Staphylococcus aureus | 4 | 12.5 | 100 |
| ON89 | | | |
| MRSA ON184 | 2 | 12.5 | 50 |
| Pseudomonas | 4 | 12.5 | 100 |
| aeruginosa SK1 | | | |
| Proteus mirabilis | 4 | 50 | 100 |
| ON153 | | | |
| Escherichia coli SK2 | 2 | 12.5 | 50 |
| Candida albicans SK4b | 1 | 12.5 | 50 |
| C. albicans ON47 | 1 | 12.5 | 25 |

TABLE 9
Fractional Biofilm Eradication Concentration Index (FBECI)
of Tetrasodium EDTA Combined with Ethanol

| Organism | EDTA (% w/v) | Ethanol (% w/v) | FBECI |
|---|---|---|---|
| Staphylococcus | 0.031 | 1.6 | 0.14 (S) |
| epidermidis SK9 | | | |
| Staphylococcus aureus | 0.063 | 0.8 | 0.08 (S) |
| ON89 | | | |
| MRSA ON184 | 0.063 | 1.6 | 0.16 (S) |
| Pseudomonas | 0.125 | 3.125 | 0.28 (S) |
| aeruginosa SK1 | | | |
| Proteus mirabilis | 0.125 | 6.25 | 0.16 (S) |
| ON153 | | | |
| Escherichia coli SK2 | 0.125 | 1.6 | 0.19 (S) |
| Candida albicans SK4b | 0.125 | 1.6 | 0.253 (S) |
| C. albicans ON47 | 0.125 | 1.6 | 0.253 (S) |

TABLE 10
Fractional Biofilm Eradication Concentration
Index (FBECI) of Tetrasodium EDTA
Combined with Cholorhexidine HCl

| | EDTA | Chlorhexidine HCl | |
|---|---|---|---|
| Organism | (% w/v) | (μg/mL) | FBECI |
| Staphylococcus | 0.125 | 3.125 | 0.19 (S) |
| epidermidis SK9 | | | |
| Staphylococcus aureus | 0.125 | 3.125 | 0.06 (S) |
| ON89 | | | |
| MRSA ON184 | 0.031 | 6.25 | 0.14 (S) |
| Pseudomonas | 0.25 | 3.125 | 0.093 (S) |
| aeruginosa SK1 | | | |
| Proteus mirabilis | 0.125 | 3.125 | 0.06 (S) |
| ON153 | | | |
| Escherichia coli SK2 | 0.031 | 3.125 | 0.08 (S) |
| Candida albicans SK4b | 0.063 | 6.25 | 0.18 (S) |
| C. albicans ON47 | 0.031 | 6.25 | 0.28 (S) |

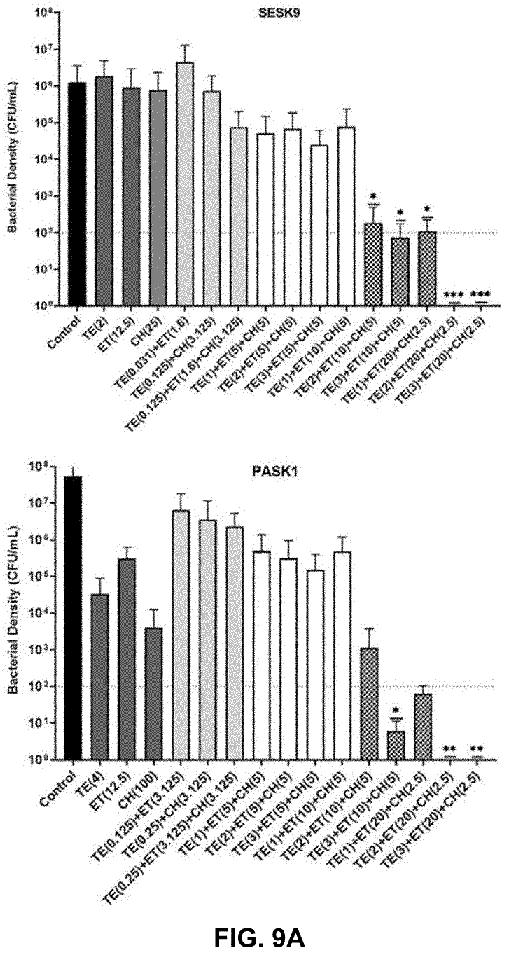

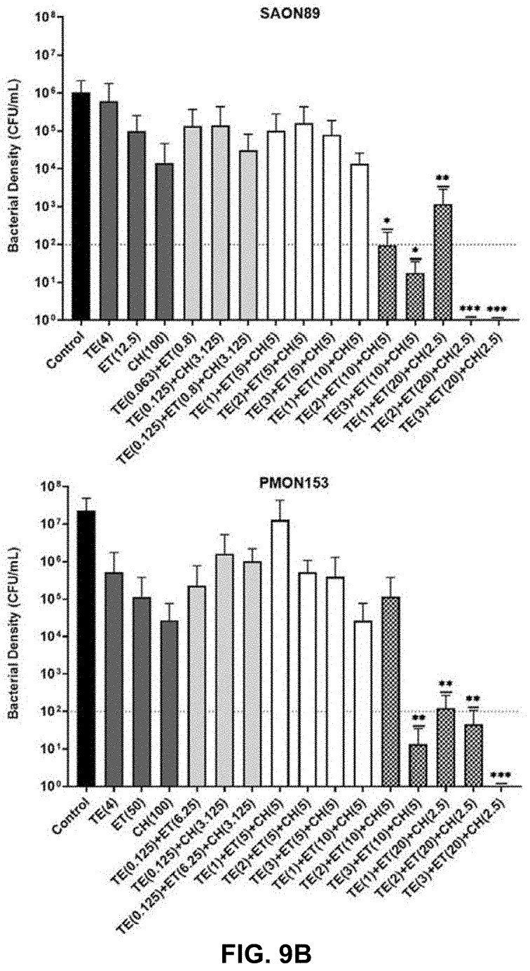

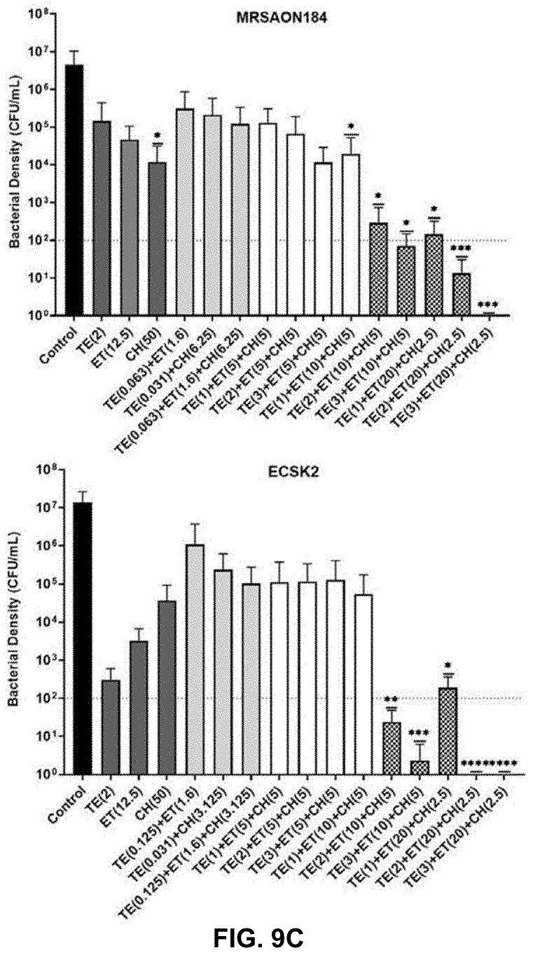

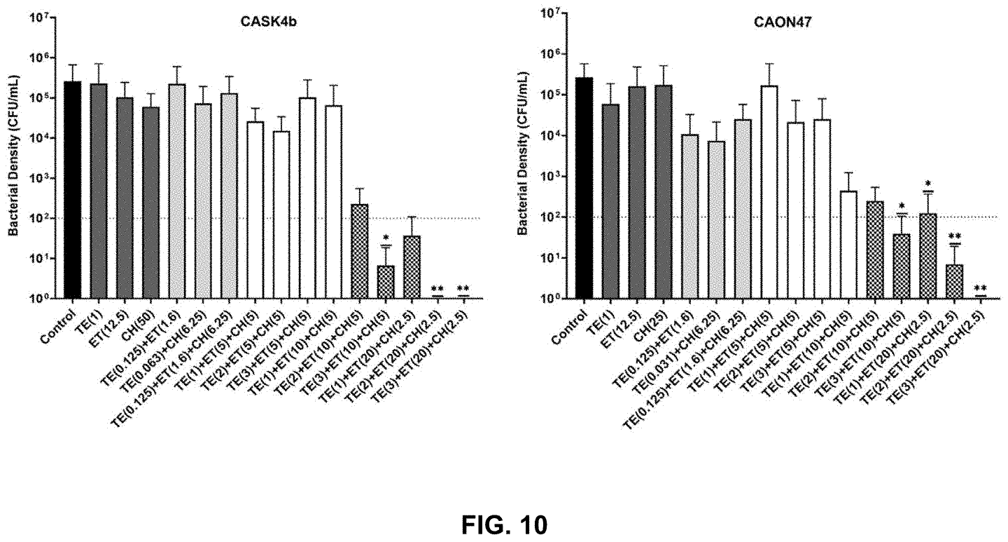

The method for rapid biofilm eradication ability of test antimicrobials alone and in combination against 48-h-old biofilms within two h exposure time included (1) choosing different concentrations of test antimicrobials to assess their potency in eradicating preformed biofilms of study organisms within two h. Then (2) the quantitative recovery from biofilms following exposure to the antimicrobial solutions for bacterial strains (FIGS. 9A-9B) and fungal strains (FIG. 5) was evaluated. Then (3) the exposure to the MBEC of tetrasodium EDTA, ethanol, or chlorhexidine HCl alone was measured as well as in several double and triple combinations of FBEC of antimicrobials failed to eradicate the preformed biofilms after two h of exposure. Then (4) all tested bacterial and fungal biofilms were entirely eradicated by the triple combination of 20% ethanol and 2.5 μg/mL chlorhexidine HCl in 3% tetrasodium EDTA (FIGS. 9A-10).

A triple combination of 20% ethanol and 2.5 μg/mL chlorhexidine HCl in 2% tetrasodium EDTA ultimately killed all biofilm cells except for three strains (MRSA ON184, P. mirabilis ON153, and C. albicans SK4b), but even for these strains, the viable cells were significantly reduced to at or near the limit of detection. Likewise, a combination of 1% tetrasodium EDTA with 20% ethanol and 2.5 μg/mL chlorhexidine HCl significantly reduced the viable cells in six of eight test organisms in comparison with their respective controls. A triple combination of 3% tetrasodium EDTA with 10% ethanol and five μg/mL chlorhexidine HCl also showed a significant reduction in viable biofilm cells of all test organisms within the 2-h contact time.

The results demonstrated that all test antimicrobials had efficient antimicrobial activity against planktonic and biofilm cells of test bacterial and fungal strains when exposed for 24 h. The combination of tetrasodium EDTA and ethanol was synergistic against planktonic cells of 6 of 12 strains tested, as measured by inhibition (FICI) and microbicidal (FMCI) activity. The interactions between tetrasodium EDTA and chlorhexidine HCl were categorized into synergistic, partially synergistic, additive, and indifferent activity against the test bacterial and fungal strains. It is noteworthy that there was no evidence of antagonistic activity between the three agents against planktonic cells in any tested combinations. We also tested the biofilm eradication ability of test antimicrobials against 48-h-old biofilms of bacterial and fungal strains within a 24-h exposure; 4% tetrasodium EDTA, 5% ethanol, and 100 μg/mL chlorhexidine HCl alone were able to eradicate all established biofilms following 24 h of treatment. As expected, biofilm cells were more resistant for each organism than planktonic cells. When tetrasodium EDTA was combined with ethanol or chlorhexidine HCl and used to treat biofilms, the agents worked synergistically, showing a remarkable reduction in concentrations compared with the MBEC values of single test antimicrobials. In many cases, the concentration of each agent required was near or lower than the MICs measured against planktonic cells. This strongly indicated that these three antimicrobials could be successfully used together to kill pathogenic microbes.

The combinations of antimicrobial agents showed efficient microbicidal activity against organisms within a reasonable contact time. Based on the results obtained from previous studies and the present study, concentrations of all three agents were chosen to optimize the effective combinations to eradicate biofilms within a selected 2-h exposure. The present study demonstrated that triple combinations of either 3% tetrasodium EDTA with 10% ethanol and 5 μg/mL chlorhexidine HCl or of 3% tetrasodium EDTA with 20% ethanol and 2.5 μg/mL chlorhexidine HCl completely eradicated 48-h-old biofilms of all of the test organisms following a 2-h exposure. In comparison with their individual antimicrobial effects, the combination of test antimicrobials significantly decreased the viable cells both of bacterial and fungal biofilms. The decrease in the ethanol concentration was compensated with an increased concentration of tetrasodium EDTA, and the effect was further accelerated with the addition of chlorhexidine HCl. The reduced ethanol concentration in the present study sets a more significant margin of safety from adverse reactions. In addition to improving safety, combination therapy may also decrease the risk of antimicrobial resistance among pathogens by reducing selection pressure. In addition, chlorhexidine concentrations above 2% have fewer human erythrocytes and neutrophils in vitro.

Additionally, toxicity of chlorhexidine is directly proportional to its concentration used. Considering this fact, the concentration of chlorhexidine HCl used in the triple combination was 0.00025% (w/v) in the present study.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present systems and methods, which, as a matter of language, might be said to fall therebetween.

What is claimed is:

1. A cleaning wipe kit, the kit comprising:
at least one first wipe, wherein the first wipe comprises a first composition comprising 1-15% (w/v) tetrasodium EDTA and ethanol, and wherein the composition has a pH of at least 9.5; and
at least one second wipe, wherein the second wipe comprises a second composition comprising 1.0-15% (w/v) tetrasodium EDTA, ethanol, and chlorhexidine, and wherein the composition has a pH of at least 9.5.

2. The cleaning wipe kit of claim 1, wherein the first composition comprises 10-60% (w/v) ethanol.

3. The cleaning wipe kit of claim 1, wherein the first composition comprises 3% (w/v) tetrasodium EDTA and 20-25% ethanol.

4. The cleaning wipe kit of claim 3, wherein the first composition comprises 20% (w/v) ethanol.

5. The cleaning wipe kit of claim 1, wherein the second composition comprises 10-60% (w/v) ethanol.

6. The cleaning wipe kit of claim 1, wherein the second composition comprises 0.5-6% (w/v) chlorhexidine.

7. The cleaning wipe kit of claim 6, wherein the second composition comprises 3% (w/v) tetrasodium EDTA, 20-25% (w/v) ethanol, and 1-2% (w/v) chlorhexidine.

8. The cleaning wipe kit of claim 7, wherein the second composition comprises 20% (w/v) ethanol.

9. The cleaning wipe kit of claim 1, wherein the at least one first wipe exudes the first composition and the at least one second wipe exudes the second composition.

10. The cleaning wipe kit of claim 1, associated with a catheter.

11. The cleaning wipe kit of claim 1, wherein the at least one first wipe is associated with insertion of a catheter.

12. The cleaning wipe kit of claim 1, wherein the at least one second wipe is associated with service of a catheter.

13. A peripheral line placement kit comprising:
a peripheral line; and
a solution chamber in communication with the peripheral line, wherein the solution chamber is configured to dispense a first disinfecting solution comprising 1-15% (w/v) tetrasodium EDTA, ethanol, chlorhexidine, and at least 1% (w/v) taurolidine, and wherein the composition has a pH of between 6.5 and 7.5 and is biocompatible in a bloodstream.

14. The kit of claim 13, further comprising at least one additional product selected from a flush solution, a lock solution, a swab, a wet wipe, and a cleaning product, wherein the additional product comprises a second disinfecting solution comprising least three of tetrasodium EDTA, ethanol, taurolidine, and chlorhexidine, and wherein a composition of the second disinfecting solution is different from a composition of the first disinfecting solution.
